SEQUENCE LISTING (159 sequences)

SEQ ID NO: 1 - protein, 30 aa, Artificial Sequence
  Artificial anti-viral peptides
KQYSVTDALEDVNTSPQSTEEVVQSFLISQ

SEQ ID NO: 2 - protein, 30 aa, Artificial Sequence
  Artificial anti-viral peptides
HQYSVTDALEDVNTSPQSTEEVVQSFLISQ

SEQ ID NO: 3 - protein, 30 aa, Artificial Sequence
  Artificial anti-viral peptides
NQYSVTDALEDVNTSPQSTEEVVQSFLISQ

SEQ ID NO: 4 - protein, 30 aa, Artificial Sequence
  Artificial anti-viral peptides
RSYSVTDALEDVNTSPQSTEEVVQSFLISQ

SEQ ID NO: 5 - protein, 30 aa, Artificial Sequence
  Artificial anti-viral peptides
RTYSVTDALEDVNTSPQSTEEVVQSFLISQ

SEQ ID NO: 6 - protein, 30 aa, Artificial Sequence
  Artificial anti-viral peptides
RQFSVTDALEDVNTSPQSTEEVVQSFLISQ

SEQ ID NO: 7 - protein, 30 aa, Artificial Sequence
  Artificial anti-viral peptides
RQWSVTDALEDVNTSPQSTEEVVQSFLISQ

SEQ ID NO: 8 - protein, 30 aa, Artificial Sequence
  Artificial anti-viral peptides
RQYTVTDALEDVNTSPQSTEEVVQSFLISQ

SEQ ID NO: 9 - protein, 30 aa, Artificial Sequence
  Artificial anti-viral peptides
RQYQVTDALEDVNTSPQSTEEVVQSFLISQ

SEQ ID NO: 10 - protein, 30 aa, Artificial Sequence
  Artificial anti-viral peptides
RQYSATDALEDVNTSPQSTEEVVQSFLISQ

SEQ ID NO: 11 - protein, 30 aa, Artificial Sequence
  Artificial anti-viral peptides
RQYSGTDALEDVNTSPQSTEEVVQSFLISQ

SEQ ID NO: 12 - protein, 30 aa, Artificial Sequence
  Artificial anti-viral peptides
RQYSVSDALEDVNTSPQSTEEVVQSFLISQ

SEQ ID NO: 13 - protein, 30 aa, Artificial Sequence
  Artificial anti-viral peptides
RQYSVQDALEDVNTSPQSTEEVVQSFLISQ

SEQ ID NO: 14 - protein, 30 aa, Artificial Sequence
  Artificial anti-viral peptides
RQYSVTEALEDVNTSPQSTEEVVQSFLISQ

SEQ ID NO: 15 - protein, 30 aa, Artificial Sequence
  Artificial anti-viral peptides
RQYSVTDGLEDVNTSPQSTEEVVQSFLISQ

SEQ ID NO: 16 - protein, 30 aa, Artificial Sequence
  Artificial anti-viral peptides
RQYSVTDVLEDVNTSPQSTEEVVQSFLISQ

SEQ ID NO: 17 - protein, 30 aa, Artificial Sequence
  Artificial anti-viral peptides
RQYSVTDAIEDVNTSPQSTEEVVQSFLISQ

SEQ ID NO: 18 - protein, 30 aa, Artificial Sequence
  Artificial anti-viral peptides
RQYSVTDALDDVNTSPQSTEEVVQSFLISQ

SEQ ID NO: 19 - protein, 30 aa, Artificial Sequence
  Artificial anti-viral peptides
RQYSVTDALEEVNTSPQSTEEVVQSFLISQ

SEQ ID NO: 20 - protein, 30 aa, Artificial Sequence
  Artificial anti-viral peptides
RQYSVTDALEDANTSPQSTEEVVQSFLISQ

SEQ ID NO: 21 - protein, 30 aa, Artificial Sequence
  Artificial anti-viral peptides
RQYSVTDALEDGNTSPQSTEEVVQSFLISQ

SEQ ID NO: 22 - protein, 30 aa, Artificial Sequence
  Artificial anti-viral peptides
RQYSVTDALEDVKTSPQSTEEVVQSFLISQ

SEQ ID NO: 23 - protein, 30 aa, Artificial Sequence
  Artificial anti-viral peptides
RQYSVTDALEDVHTSPQSTEEVVQSFLISQ

SEQ ID NO: 24 - protein, 30 aa, Artificial Sequence
  Artificial anti-viral peptides
RQYSVTDALEDVRTSPQSTEEVVQSFLISQ

SEQ ID NO: 25 - protein, 30 aa, Artificial Sequence
  Artificial anti-viral peptides
RQYSVTDALEDVNSSPQSTEEVVQSFLISQ

SEQ ID NO: 26 - protein, 30 aa, Artificial Sequence
  Artificial anti-viral peptides
RQYSVTDALEDVNQSPQSTEEVVQSFLISQ

SEQ ID NO: 27 - protein, 30 aa, Artificial Sequence
  Artificial anti-viral peptides
RQYSVTDALEDVNTTPQSTEEVVQSFLISQ

SEQ ID NO: 28 - protein, 30 aa, Artificial Sequence
  Artificial anti-viral peptides
RQYSVTDALEDVNTQPQSTEEVVQSFLISQ

SEQ ID NO: 29 - protein, 30 aa, Artificial Sequence
  Artificial anti-viral peptides
RQYSVTDALEDVNTSPSSTEEVVQSFLISQ

SEQ ID NO: 30 - protein, 30 aa, Artificial Sequence
  Artificial anti-viral peptides
RQYSVTDALEDVNTSPTSTEEVVQSFLISQ

SEQ ID NO: 31 - protein, 30 aa, Artificial Sequence
  Artificial anti-viral peptides
RQYSVTDALEDVNTSPQTTEEVVQSFLISQ

SEQ ID NO: 32 - protein, 30 aa, Artificial Sequence
  Artificial anti-viral peptides
RQYSVTDALEDVNTSPQQTEEVVQSFLISQ

SEQ ID NO: 33 - protein, 30 aa, Artificial Sequence
  Artificial anti-viral peptides
RQYSVTDALEDVNTSPQSSEEVVQSFLISQ

SEQ ID NO: 34 - protein, 30 aa, Artificial Sequence
  Artificial anti-viral peptides
RQYSVTDALEDVNTSPQSQEEVVQSFLISQ

SEQ ID NO: 35 - protein, 30 aa, Artificial Sequence
  Artificial anti-viral peptides
RQYSVTDALEDVNTSPQSTDEVVQSFLISQ

SEQ ID NO: 36 - protein, 30 aa, Artificial Sequence
  Artificial anti-viral peptides
RQYSVTDALEDVNTSPQSTEDVVQSFLISQ

SEQ ID NO: 37 - protein, 30 aa, Artificial Sequence
  Artificial anti-viral peptides
RQYSVTDALEDVNTSPQSTEEAVQSFLISQ

SEQ ID NO: 38 - protein, 30 aa, Artificial Sequence
  Artificial anti-viral peptides
RQYSVTDALEDVNTSPQSTEEGVQSFLISQ

SEQ ID NO: 39 - protein, 30 aa, Artificial Sequence
  Artificial anti-viral peptides
RQYSVTDALEDVNTSPQSTEEVAQSFLISQ

SEQ ID NO: 40 - protein, 30 aa, Artificial Sequence
  Artificial anti-viral peptides
RQYSVTDALEDVNTSPQSTEEVGQSFLISQ

SEQ ID NO: 41 - protein, 30 aa, Artificial Sequence
  Artificial anti-viral peptides
RQYSVTDALEDVNTSPQSTEEVVSSFLISQ

SEQ ID NO: 42 - protein, 30 aa, Artificial Sequence
  Artificial anti-viral peptides
RQYSVTDALEDVNTSPQSTEEVVTSFLISQ

SEQ ID NO: 43 - protein, 30 aa, Artificial Sequence
  Artificial anti-viral peptides
RQYSVTDALEDVNTSPQSTEEVVQTFLISQ

SEQ ID NO: 44 - protein, 30 aa, Artificial Sequence
  Artificial anti-viral peptides
RQYSVTDALEDVNTSPQSTEEVVQQFLISQ

SEQ ID NO: 45 - protein, 30 aa, Artificial Sequence
  Artificial anti-viral peptides
RQYSVTDALEDVNTSPQSTEEVVQSWLISQ

SEQ ID NO: 46 - protein, 30 aa, Artificial Sequence
  Artificial anti-viral peptides
RQYSVTDALEDVNTSPQSTEEVVQSYLISQ

SEQ ID NO: 47 - protein, 30 aa, Artificial Sequence
  Artificial anti-viral peptides
RQYSVTDALEDVNTSPQSTEEVVQSFIISQ

SEQ ID NO: 48 - protein, 30 aa, Artificial Sequence
  Artificial anti-viral peptides
RQYSVTDALEDVNTSPQSTEEVVQSFLLSQ

SEQ ID NO: 49 - protein, 30 aa, Artificial Sequence
  Artificial anti-viral peptides
RQYSVTDALEDVNTSPQSTEEVVQSFLITQ

SEQ ID NO: 50 - protein, 30 aa, Artificial Sequence
  Artificial anti-viral peptides
RQYSVTDALEDVNTSPQSTEEVVQSFLIQQ

SEQ ID NO: 51 - protein, 30 aa, Artificial Sequence
  Artificial anti-viral peptides
RQYSVTDALEDVNTSPQSTEEVVQSFLISS

SEQ ID NO: 52 - protein, 30 aa, Artificial Sequence
  Artificial anti-viral peptides
RQYSVTDALEDVNTSPQSTEEVVQSFLIST

SEQ ID NO: 53 - protein, 30 aa, Artificial Sequence
  Artificial anti-viral peptides
RQYSVTDALEDVNTSPQSTEEVVQSFLISQ

SEQ ID NO: 54 - protein, 30 aa, Artificial Sequence
  Artificial anti-viral peptides
RQYSVTDALEDVSTSPQSTEEVVQSFLISQ

SEQ ID NO: 55 - protein, 30 aa, Artificial Sequence
  Artificial anti-viral peptides
GDVDVSAVQAKLGALELNQRDAAAETELRV

SEQ ID NO: 56 - protein, 30 aa, Artificial Sequence
  Artificial anti-viral peptides
VDVDVSAVQAKLGALELNQRDAAAETELRV

SEQ ID NO: 57 - protein, 30 aa, Artificial Sequence
  Artificial anti-viral peptides
AEVDVSAVQAKLGALELNQRDAAAETELRV

SEQ ID NO: 58 - protein, 30 aa, Artificial Sequence
  Artificial anti-viral peptides
ADADVSAVQAKLGALELNQRDAAAETELRV

SEQ ID NO: 59 - protein, 30 aa, Artificial Sequence
  Artificial anti-viral peptides
ADGDVSAVQAKLGALELNQRDAAAETELRV

SEQ ID NO: 60 - protein, 30 aa, Artificial Sequence
  Artificial anti-viral peptides
ADVEVSAVQAKLGALELNQRDAAAETELRV

SEQ ID NO: 61 - protein, 30 aa, Artificial Sequence
  Artificial anti-viral peptides
ADVDASAVQAKLGALELNQRDAAAETELRV

SEQ ID NO: 62 - protein, 30 aa, Artificial Sequence
  Artificial anti-viral peptides
ADVDGSAVQAKLGALELNQRDAAAETELRV

SEQ ID NO: 63 - protein, 30 aa, Artificial Sequence
  Artificial anti-viral peptides
ADVDVTAVQAKLGALELNQRDAAAETELRV

SEQ ID NO: 64 - protein, 30 aa, Artificial Sequence
  Artificial anti-viral peptides
ADVDVQAVQAKLGALELNQRDAAAETELRV

SEQ ID NO: 65 - protein, 30 aa, Artificial Sequence
  Artificial anti-viral peptides
ADVDVSGVQAKLGALELNQRDAAAETELRV

SEQ ID NO: 66 - protein, 30 aa, Artificial Sequence
  Artificial anti-viral peptides
ADVDVSVVQAKLGALELNQRDAAAETELRV

SEQ ID NO: 67 - protein, 30 aa, Artificial Sequence
  Artificial anti-viral peptides
ADVDVSAAQAKLGALELNQRDAAAETELRV

SEQ ID NO: 68 - protein, 30 aa, Artificial Sequence
  Artificial anti-viral peptides
ADVDVSAGQAKLGALELNQRDAAAETELRV

SEQ ID NO: 69 - protein, 30 aa, Artificial Sequence
  Artificial anti-viral peptides
ADVDVSAVSAKLGALELNQRDAAAETELRV

SEQ ID NO: 70 - protein, 30 aa, Artificial Sequence
  Artificial anti-viral peptides
ADVDVSAVTAKLGALELNQRDAAAETELRV

SEQ ID NO: 71 - protein, 30 aa, Artificial Sequence
  Artificial anti-viral peptides
ADVDVSAVQGKLGALELNQRDAAAETELRV

SEQ ID NO: 72 - protein, 30 aa, Artificial Sequence
  Artificial anti-viral peptides
ADVDVSAVQVKLGALELNQRDAAAETELRV

SEQ ID NO: 73 - protein, 30 aa, Artificial Sequence
  Artificial anti-viral peptides
ADVDVSAVQAHLGALELNQRDAAAETELRV

SEQ ID NO: 74 - protein, 30 aa, Artificial Sequence
  Artificial anti-viral peptides
ADVDVSAVQARLGALELNQRDAAAETELRV

SEQ ID NO: 75 - protein, 30 aa, Artificial Sequence
  Artificial anti-viral peptides
ADVDVSAVQANLGALELNQRDAAAETELRV

SEQ ID NO: 76 - protein, 30 aa, Artificial Sequence
  Artificial anti-viral peptides
ADVDVSAVQAKIGALELNQRDAAAETELRV

SEQ ID NO: 77 - protein, 30 aa, Artificial Sequence
  Artificial anti-viral peptides
ADVDVSAVQAKLAALELNQRDAAAETELRV

SEQ ID NO: 78 - protein, 30 aa, Artificial Sequence
  Artificial anti-viral peptides
ADVDVSAVQAKLVALELNQRDAAAETELRV

SEQ ID NO: 79 - protein, 30 aa, Artificial Sequence
  Artificial anti-viral peptides
ADVDVSAVQAKLGGLELNQRDAAAETELRV

SEQ ID NO: 80 - protein, 30 aa, Artificial Sequence
  Artificial anti-viral peptides
ADVDVSAVQAKLGVLELNQRDAAAETELRV

SEQ ID NO: 81 - protein, 30 aa, Artificial Sequence
  Artificial anti-viral peptides
ADVDVSAVQAKLGAIELNQRDAAAETELRV

SEQ ID NO: 82 - protein, 30 aa, Artificial Sequence
  Artificial anti-viral peptides
ADVDVSAVQAKLGALDLNQRDAAAETELRV

SEQ ID NO: 83 - protein, 30 aa, Artificial Sequence
  Artificial anti-viral peptides
ADVDVSAVQAKLGALEINQRDAAAETELRV

SEQ ID NO: 84 - protein, 30 aa, Artificial Sequence
  Artificial anti-viral peptides
ADVDVSAVQAKLGALELKQRDAAAETELRV

SEQ ID NO: 85 - protein, 30 aa, Artificial Sequence
  Artificial anti-viral peptides
ADVDVSAVQAKLGALELHQRDAAAETELRV

SEQ ID NO: 86 - protein, 30 aa, Artificial Sequence
  Artificial anti-viral peptides
ADVDVSAVQAKLGALELRQRDAAAETELRV

SEQ ID NO: 87 - protein, 30 aa, Artificial Sequence
  Artificial anti-viral peptides
ADVDVSAVQAKLGALELNSRDAAAETELRV

SEQ ID NO: 88 - protein, 30 aa, Artificial Sequence
  Artificial anti-viral peptides
ADVDVSAVQAKLGALELNTRDAAAETELRV

SEQ ID NO: 89 - protein, 30 aa, Artificial Sequence
  Artificial anti-viral peptides
ADVDVSAVQAKLGALELNQKDAAAETELRV

SEQ ID NO: 90 - protein, 30 aa, Artificial Sequence
  Artificial anti-viral peptides
ADVDVSAVQAKLGALELNQHDAAAETELRV

SEQ ID NO: 91 - protein, 30 aa, Artificial Sequence
  Artificial anti-viral peptide
ADVDVSAVQAKLGALELNQNDAAAETELRV

SEQ ID NO: 92 - protein, 30 aa, Artificial Sequence
  Artificial anti-viral peptide
ADVDVSAVQAKLGALELNQREAAAETELRV

SEQ ID NO: 93 - protein, 30 aa, Artificial Sequence
  Artificial anti-viral peptide
ADVDVSAVQAKLGALELNQRDGAAETELRV

SEQ ID NO: 94 - protein, 30 aa, Artificial Sequence
  Artificial anti-viral peptide
ADVDVSAVQAKLGALELNQRDVAAETELRV

SEQ ID NO: 95 - protein, 30 aa, Artificial Sequence
  Artificial anti-viral peptide
ADVDVSAVQAKLGALELNQRDAGAETELRV

SEQ ID NO: 96 - protein, 30 aa, Artificial Sequence
  Artificial anti-viral peptide
ADVDVSAVQAKLGALELNQRDAVAETELRV

SEQ ID NO: 97 - protein, 30 aa, Artificial Sequence
  Artificial anti-viral peptide
ADVDVSAVQAKLGALELNQRDAAGETELRV

SEQ ID NO: 98 - protein, 30 aa, Artificial Sequence
  Artificial anti-viral peptide
ADVDVSAVQAKLGALELNQRDAAVETELRV

SEQ ID NO: 99 - protein, 30 aa, Artificial Sequence
  Artificial anti-viral peptide
ADVDVSAVQAKLGALELNQRDAAADTELRV

SEQ ID NO: 100 - protein, 30 aa, Artificial Sequence
  Artificial anti-viral peptide
ADVDVSAVQAKLGALELNQRDAAAESELRV

SEQ ID NO: 101 - protein, 30 aa, Artificial Sequence
  Artificial anti-viral peptide
ADVDVSAVQAKLGALELNQRDAAAEQELRV

SEQ ID NO: 102 - protein, 30 aa, Artificial Sequence
  Artificial anti-viral peptide
ADVDVSAVQAKLGALELNQRDAAAETDLRV

SEQ ID NO: 103 - protein, 30 aa, Artificial Sequence
  Artificial anti-viral peptide
ADVDVSAVQAKLGALELNQRDAAAETEIRV

SEQ ID NO: 104 - protein, 30 aa, Artificial Sequence
  Artificial anti-viral peptide
ADVDVSAVQAKLGALELNQRDAAAETELKV

SEQ ID NO: 105 - protein, 30 aa, Artificial Sequence
  Artificial anti-viral peptide
ADVDVSAVQAKLGALELNQRDAAAETELHV

SEQ ID NO: 106 - protein, 30 aa, Artificial Sequence
  Artificial anti-viral peptide
ADVDVSAVQAKLGALELNQRDAAAETELNV

SEQ ID NO: 107 - protein, 30 aa, Artificial Sequence
  Artificial anti-viral peptide
ADVDVSAVQAKLGALELNQRDAAAETELRA

SEQ ID NO: 108 - protein, 30 aa, Artificial Sequence
  Artificial anti-viral peptide
ADVDVSAVQAKLGALELNQRDAAAETELRG

SEQ ID NO: 109 - protein, 30 aa, Artificial Sequence
  Artificial anti-viral peptide
ADVDVSAVQAKLGALELNQRDAAAETELRV

SEQ ID NO: 110 - protein, 25 aa, Artificial Sequence
  Artificial anti-viral peptide
ADTVGLIDEQNEASKTNGLGAAEAF

SEQ ID NO: 111 - protein, 25 aa, Artificial Sequence
  Artificial anti-viral peptide
VDTVGLIDEQNEASKTNGLGAAEAF

SEQ ID NO: 112 - protein, 25 aa, Artificial Sequence
  Artificial anti-viral peptide
GETVGLIDEQNEASKTNGLGAAEAF

SEQ ID NO: 113 - protein, 25 aa, Artificial Sequence
  Artificial anti-viral peptide
GDSVGLIDEQNEASKTNGLGAAEAF

SEQ ID NO: 114 - protein, 25 aa, Artificial Sequence
  Artificial anti-viral peptide
GDQVGLIDEQNEASKTNGLGAAEAF

SEQ ID NO: 115 - protein, 25 aa, Artificial Sequence
  Artificial anti-viral peptide
GDTAGLIDEQNEASKTNGLGAAEAF

SEQ ID NO: 116 - protein, 25 aa, Artificial Sequence
  Artificial anti-viral peptide
GDTGGLIDEQNEASKTNGLGAAEAF

SEQ ID NO: 117 - protein, 25 aa, Artificial Sequence
  Artificial anti-viral peptide
GDTVALIDEQNEASKTNGLGAAEAF

SEQ ID NO: 118 - protein, 25 aa, Artificial Sequence
  Artificial anti-viral peptide
GDTVVLIDEQNEASKTNGLGAAEAF

SEQ ID NO: 119 - protein, 25 aa, Artificial Sequence
  Artificial anti-viral peptide
GDTVGIIDEQNEASKTNGLGAAEAF

SEQ ID NO: 120 - protein, 25 aa, Artificial Sequence
  Artificial anti-viral peptide
GDTVGLLDEQNEASKTNGLGAAEAF

SEQ ID NO: 121 - protein, 25 aa, Artificial Sequence
  Artificial anti-viral peptide
GDTVGLIEEQNEASKTNGLGAAEAF

SEQ ID NO: 122 - protein, 25 aa, Artificial Sequence
  Artificial anti-viral peptide
GDTVGLIDDQNEASKTNGLGAAEAF

SEQ ID NO: 123 - protein, 25 aa, Artificial Sequence
  Artificial anti-viral peptide
GDTVGLIDESNEASKTNGLGAAEAF

SEQ ID NO: 124 - protein, 25 aa, Artificial Sequence
  Artificial anti-viral peptide
GDTVGLIDETNEASKTNGLGAAEAF

SEQ ID NO: 125 - protein, 25 aa, Artificial Sequence
  Artificial anti-viral peptide
GDTVGLIDEQKEASKTNGLGAAEAF

SEQ ID NO: 126 - protein, 25 aa, Artificial Sequence
  Artificial anti-viral peptide
GDTVGLIDEQHEASKTNGLGAAEAF

SEQ ID NO: 127 - protein, 25 aa, Artificial Sequence
  Artificial anti-viral peptide
GDTVGLIDEQREASKTNGLGAAEAF

SEQ ID NO: 128 - protein, 25 aa, Artificial Sequence
  Artificial anti-viral peptide
GDTVGLIDEQNDASKTNGLGAAEAF

SEQ ID NO: 129 - protein, 25 aa, Artificial Sequence
  Artificial anti-viral peptide
GDTVGLIDEQNEGSKTNGLGAAEAF

SEQ ID NO: 130 - protein, 25 aa, Artificial Sequence
  Artificial anti-viral peptide
GDTVGLIDEQNEVSKTNGLGAAEAF

SEQ ID NO: 131 - protein, 25 aa, Artificial Sequence
  Artificial anti-viral peptide
GDTVGLIDEQNEATKTNGLGAAEAF

SEQ ID NO: 132 - protein, 25 aa, Artificial Sequence
  Artificial anti-viral peptide
GDTVGLIDEQNEAQKTNGLGAAEAF

SEQ ID NO: 133 - protein, 25 aa, Artificial Sequence
  Artificial anti-viral peptide
GDTVGLIDEQNEASHTNGLGAAEAF

SEQ ID NO: 134 - protein, 25 aa, Artificial Sequence
  Artificial anti-viral peptide
GDTVGLIDEQNEASRTNGLGAAEAF

SEQ ID NO: 135 - protein, 25 aa, Artificial Sequence
  Artificial anti-viral peptide
GDTVGLIDEQNEASNTNGLGAAEAF

SEQ ID NO: 136 - protein, 25 aa, Artificial Sequence
  Artificial anti-viral peptide
GDTVGLIDEQNEASKSNGLGAAEAF

SEQ ID NO: 137 - protein, 25 aa, Artificial Sequence
  Artificial anti-viral peptide
GDTVGLIDEQNEASKQNGLGAAEAF

SEQ ID NO: 138 - protein, 25 aa, Artificial Sequence
  Artificial anti-viral peptide
GDTVGLIDEQNEASKTKGLGAAEAF

SEQ ID NO: 139 - protein, 25 aa, Artificial Sequence
  Artificial anti-viral peptide
GDTVGLIDEQNEASKTHGLGAAEAF

SEQ ID NO: 140 - protein, 25 aa, Artificial Sequence
  Artificial anti-viral peptide
GDTVGLIDEQNEASKTRGLGAAEAF

SEQ ID NO: 141 - protein, 25 aa, Artificial Sequence
  Artificial anti-viral peptide
GDTVGLIDEQNEASKTNALGAAEAF

SEQ ID NO: 142 - protein, 25 aa, Artificial Sequence
  Artificial anti-viral peptide
GDTVGLIDEQNEASKTNVLGAAEAF

SEQ ID NO: 143 - protein, 25 aa, Artificial Sequence
  Artificial anti-viral peptide
GDTVGLIDEQNEASKTNGIGAAEAF

SEQ ID NO: 144 - protein, 25 aa, Artificial Sequence
  Artificial anti-viral peptide
GDTVGLIDEQNEASKTNGLAAAEAF

SEQ ID NO: 145 - protein, 25 aa, Artificial Sequence
  Artificial anti-viral peptide
GDTVGLIDEQNEASKTNGLVAAEAF

SEQ ID NO: 146 - protein, 25 aa, Artificial Sequence
  Artificial anti-viral peptide
GDTVGLIDEQNEASKTNGLGGAEAF

SEQ ID NO: 147 - protein, 25 aa, Artificial Sequence
  Artificial anti-viral peptide
GDTVGLIDEQNEASKTNGLGVAEAF

SEQ ID NO: 148 - protein, 25 aa, Artificial Sequence
  Artificial anti-viral peptide
GDTVGLIDEQNEASKTNGLGAGEAF

SEQ ID NO: 149 - protein, 25 aa, Artificial Sequence
  Artificial anti-viral peptide
GDTVGLIDEQNEASKTNGLGAVEAF

SEQ ID NO: 150 - protein, 25 aa, Artificial Sequence
  Artificial anti-viral peptide
GDTVGLIDEQNEASKTNGLGAADAF

SEQ ID NO: 151 - protein, 25 aa, Artificial Sequence
  Artificial anti-viral peptide
GDTVGLIDEQNEASKTNGLGAAEGF

SEQ ID NO: 152 - protein, 25 aa, Artificial Sequence
  Artificial anti-viral peptide
GDTVGLIDEQNEASKTNGLGAAEVF

SEQ ID NO: 153 - protein, 25 aa, Artificial Sequence
  Artificial anti-viral peptide
GDTVGLIDEQNEASKTNGLGAAEAW

SEQ ID NO: 154 - protein, 25 aa, Artificial Sequence
  Artificial anti-viral peptide
GDTVGLIDEQNEASKTNGLGAAEAY

SEQ ID NO: 155 - protein, 25 aa, Artificial Sequence
  Artificial anti-viral peptide
GDTVGLIDEQNEASKTNGLGAAEAF

SEQ ID NO: 156 - protein, 30 aa, Artificial Sequence
  Artificial anti-viral peptide
  VARIANT 1: Xaa = Arg, Lys, His, Asn, Glu, Asp, or Gln
  VARIANT 2: Xaa = Gln, Arg, Glu, His, Lys, Ser, Thr or Cys
  VARIANT 3: Xaa = Tyr, His, Phe, or Trp
  VARIANT 4: Xaa = Ser, Ala, Asn, Thr, Cys, or Gln
  VARIANT 5: Xaa = Val, Ile, Leu, Met, Gly, Ala, or Leu
  VARIANT (6)...(6): Xaa = Thr, Ser Cys or Gln
  VARIANT (7)...(7): Xaa = Asp, Asn, Glu, Lys or Arg
  VARIANT (8)...(8): Xaa = Gly, Ala, Val, Leu, Met, Ile or Ser
  VARIANT (9)...(9): Xaa = Leu, Ile, Met, Phe, Val, Gly or Ala
  VARIANT (10)...(10): Xaa = Glu, Asp, Gln, Lys, His Arg, or Asn
  VARIANT (11)...(11): Xaa = Asp, Asn, Glu, Lys or Arg
  VARIANT (12)...(12): Xaa = Tyr, His, Phe or Trp
  VARIANT (13)...(13): Xaa = Asn, Asp, His, Ser, Lys, Arg, or Glu
  VARIANT (14)...(14): Xaa =  Thr, Ser, Cys or Gln
  VARIANT (15)...(15): Xaa = Ser, Ala, Asn, Thr, Cys or Gln
  VARIANT (17)...(17): Xaa = Gln, Arg, Glu, His, Lys, Ser, Thr or Cys
  VARIANT (18)...(18): Xaa = Ser, Ala, Asn, Thr, Cys or Gln
  VARIANT 19: Xaa = Thr, Ser, Cys or Gln
  VARIANT 20: Xaa = Glu, Asp, Gln, Lys, His, Arg or Asn
  VARIANT 21: Xaa = Glu, Asp, Gln, Lys, His, Arg or Asn
  VARIANT 22: Xaa = Val, Ile, Leu, Met, Gly, Ala or Leu
  VARIANT 23: Xaa = Val, Ile, Leu, Met, Gly, Ala or Leu
  VARIANT 24: Xaa = Gln, Arg, Glu, His, Lys, Ser, Thr or Cys
  VARIANT 25: Xaa = Ser, Ala, Asn, Thr, Cys or Gln
  VARIANT 26: Xaa = Phe, Leu, Trp or Tyr
  VARIANT 27: Xaa = Leu, Ile, Met, Phe, Val, Gly or Ala
  VARIANT 28: Xaa = Ile, Leu, Met, Val, Gly or Ala
  VARIANT 29: Xaa = Ser, Ala, Asn, Thr, Cys or Gln
  VARIANT 30: Xaa = Gln, Arg, Glu, His, Lys, Ser, Thr or Cys
XXXXXXXXXXXXXXXPXXXXXXXXXXXXXX

SEQ ID NO: 157 - protein, 30 aa, Artificial Sequence; identical to SEQ ID NO: 156
  Artificial anti-viral peptide
  VARIANT 1: Xaa = Arg, Lys, His, Asn, Glu, Asp, or Gln
  VARIANT 2: Xaa = Gln, Arg, Glu, His, Lys, Ser, Thr or Cys
  VARIANT 3: Xaa = Tyr, His, Phe or Trp
  VARIANT 4: Xaa = Ser, Ala, Asn, Thr, Cys or Gln
  VARIANT 5: Xaa = Val, Ile Leu, Met, Gly, Ala or Leu
  VARIANT 6: Xaa = Thr, Ser, Cys or Gln
  VARIANT 7: Xaa = Asp, Asn, Glu, Lys or Arg
  VARIANT 8: Xaa = Gly, Ala, Val, Leu, Met, Ile or Ser
  VARIANT 9: Xaa = Leu, Ile, Met, Phe, Val, Gly or Ala
  VARIANT 10: Xaa = Glu, Asp, Gln, Lys, His, Arg or Asn
  VARIANT 11: Xaa = Asp, Asn, Glu, Lys or Arg
  VARIANT 12: Xaa = Tyr, His, Phe or Trp
  VARIANT 13: Xaa = Ser, Ala, Asn, Thr, Cys or Gln
  VARIANT 14: Xaa = Thr, Ser, Cys or Gln
  VARIANT 15: Xaa = Ser, Ala, Asn, Thr, Cys or Gln
  VARIANT 17: Xaa = Gln, Arg, Glu, His, Lys, Ser, Thr or Cys
  VARIANT 18: Xaa = Ser, Ala, Asn, Thr, Cys or Gln
  VARIANT 19: Xaa = Thr, Ser, Cys or Gln
  VARIANT 20: Xaa = Glu, Asp, Gln, Lys, His, Arg, Asn
  VARIANT 21: Xaa = Val, Ile Leu, Met, Gly, Ala or Leu
  VARIANT 22: Xaa = Val, Ile, Leu, Met, Gly Ala or Leu
  VARIANT 23: Xaa = Val, Ile, Leu, Met Gly, Ala, or Leu
  VARIANT 24: Xaa = Gln, Arg, Glu, His, Lys, Ser, Thr or Cys
  VARIANT 25: Xaa = Ser, Ala, Asn, Thr, Cys or Gln
  VARIANT 26: Xaa = Phe, Leu, Trp, or Tyr
  VARIANT 27: Xaa = Leu, Ile, Met, Phe, Val, Gly or Ala
  VARIANT 28: Xaa = Ile, Leu, Met, Val Gly or Ala
  VARIANT 29: Xaa = Ser, Ala, Asn, Thr, Cys or Gln
  VARIANT 30: Xaa = Gln, Arg, Glu, His, Lys, Ser, Thr or Cys

SEQ ID NO: 158 - protein, 30 aa, Artificial Sequence
  Artificial anti-viral peptide
  VARIANT 1: Xaa = Ala, Gly, Ala, Val, Leu, Met, Ile, or Ser
  VARIANT 2: Xaa = Asp, Asn, Glu, Lys or Arg
  VARIANT 3: Xaa = Val, Ile, Leu, Met, Gly, Ala or Leu
  VARIANT 4: Xaa = Asp, Asn, Glu, Lys or Arg
  VARIANT 5: Xaa = Val, Ile, Leu, Met, Gly, Ala or Leu
  VARIANT 6: Xaa = Ser, Ala, Asn, Thr, Cys or Gln
  VARIANT 7: Xaa = Ala, Gly, Ala, Val, Leu, Met, Ile or Ser
  VARIANT 8: Xaa = Val, Ile, Leu, Met Gly, Ala or Leu
  VARIANT 9: Xaa = Gln, Arg, Glu, His, Lys, Ser, Thr or Cys
  VARIANT 10: Xaa = Ala, Gly, Ala, Val, Leu, Met, Ile or Ser
  VARIANT 11: Xaa = Lys, Arg, Glu, Gln, His, Asn or Asp
  VARIANT 12: Xaa = Leu, Ile, Met, Phe, Val, Gly or Ala
  VARIANT 13: Xaa = Gly, Ala, Val, Leu, Met or Ile
  VARIANT 14: Xaa = Ala, Gly, Ala, Val, Leu, Met, Ile or Ser
  VARIANT 15: Xaa = Leu, Ile, Met, Phe, Val, Gly or Ala
  VARIANT 16: Xaa = Glu, Asp, Gln, Lys, His Arg or Asn
  VARIANT 17: Xaa = Leu, Ile, Met, Phe, Val, Gly or Ala
  VARIANT 18: Xaa = Asn, Asp, His, Ser, Lys, Arg or Glu
  VARIANT 19: Xaa = Gln, Arg, Glu, His, Lys, Ser, Thr or Cys
  VARIANT 20: Xaa = Arg, Lys, His, Asn, Glu, Asp, or Gln
  VARIANT 21: Xaa = Asp, Asn, Glu, Lys or Arg
  VARIANT 22: Xaa = Ala, Gly, Ala, Val, Leu, Met, Ile or Ser
  VARIANT 23: Xaa = Ala, Gly, Ala, Val, Leu, Met, Ile, or Ser
  VARIANT 24: Xaa = Ala, Gly, Ala, Val, Leu, Met, Ile, or Ser
  VARIANT 25: Xaa = Glu, Asp, Gln, Lys, His, Arg or Asn
  VARIANT 26: Xaa = Thr, Ser, Cys or Gln
  VARIANT 27: Xaa = Glu, Asp, Gln, Lys, His, Arg or Asn
  VARIANT 28: Xaa = Leu, Ile, Met, Phe, Val, Gly or Ala
  VARIANT 29: Xaa = Arg, Lys, His, Asn, Glu, Asp or Gln
  VARIANT 30: Xaa = Val, Ile, Leu, Met, Gly, Ala, or Leu
XXXXXXXXXXXXXXXXXXXXXXXXXXXXXX

SEQ ID NO: 159 - protein, 25 aa, Artificial Sequence
  Artificial anti-viral peptide
  VARIANT 1: Xaa = Gly, Ala, Val, Leu, Met or Ile
  VARIANT 2: Xaa = Asp, Asn, Glu, Lys or Arg
  VARIANT 3: Xaa = Thr, Ser, Cys or Gln
  VARIANT 4: Xaa = Val, Ile, Leu, Met, Gly, Ala or Leu
  VARIANT 5: Xaa = Gly, Ala, Val, Leu, Met, or Ile
  VARIANT 6: Xaa = Leu, Ile, Met, Phe, Val, Gly or Ala
  VARIANT 7: Xaa = Ile, Leu, Met, Val, Gly or Ala
  VARIANT 8: Xaa = Asp, Asn, Glu, Lys or Arg
  VARIANT 9: Xaa = Glu, Asp, Gln, Lys, His, Arg or Asn
  VARIANT 10: Xaa = Gln, Arg, Glu, His, Lys, Ser, Thr or Cys
  VARIANT 11: Xaa = Asn, Asp, His, Ser, Lys, Arg or Glu
  VARIANT 12: Xaa = Glu, Asp, Gln, Lys, His, Arg or Asn
  VARIANT 13: Xaa = Ala, Gly, Ala, Val, Leu, Met, Ile or Ser
  VARIANT 14: Xaa = Ser, Ala, Asn, Thr, Cys or Gln
  VARIANT 15: Xaa = Lys, Arg, Glu, Gln, His, Asn or Asp
  VARIANT 16: Xaa = Thr, Ser, Cys or Gln
  VARIANT 17: Xaa = Asn, Asp, His, Ser, Lys, Arg or Glu
  VARIANT 18: Xaa = Gly, Ala, Val, Leu, Met or Ile
  VARIANT 19: Xaa = Leu, Ile, Met, Phe, Val, Gly, or Ala
  VARIANT 20: Xaa = Gly, Ala, Val, Leu, Met or Ile
  VARIANT 21: Xaa = Ala, Gly, Ala, Val, Leu, Met, Ile or Ser
  VARIANT 22: Xaa = Ala, Gly, Ala, Val, Leu, Met, Ile or Ser
  VARIANT 23: Xaa = Glu, Asp, Gln, Lys, His, Arg or Asn
  VARIANT 24: Xaa = Ala, Gly, Ala, Val, Leu, Met, Ile or Ser
  VARIANT 25: Xaa = Phe, Leu, Trp or Tyr
XXXXXXXXXXXXXXXXXXXXXXXXX

(19) United States
(12) Patent Application Publication
(10) Pub. No.: US 2017/0298100 A1
(43) Pub. Date: Oct. 19, 2017

(54) ANTI-VIRAL PEPTIDES

(71) Applicant: Cascadia Life Sciences LLC, Seattle, WA (US)

(72) Inventor: Robert J. Livingston, Seattle, WA (US)

(21) Appl. No.: 15/515,971

(22) PCT Filed: Sep. 30, 2015
(86) PCT No.: PCT/US15/53289

§ 371 Date: Mar. 30, 2017

Related U.S. Application Data
(60) Provisional application No. 62/058,557, filed on Oct. 1, 2014.

(51) Int. Cl. C07K 14/00; A61K 38/00

(52) U.S. Cl. CPC C07K 14/00; A61K 38/00; C07K 2319/00

(57) ABSTRACT

Novel antiviral polypeptides are disclosed along with methods for their use to interfere with viral replication cycles by substantially impairing the binding of viruses to target cells, viral replication and assembly in infected cells, and viral egress from infected cells including viral lysis of host cells. The present antiviral peptides exhibit broad specificity across a range of human viral pathogens by virtue of their derivation from selected viral resistance genes and their ability to interfere with conserved mechanisms of host cell-virus interactions.

### CROSS REFERENCE TO RELATED APPLICATION

This application claims the benefit under 35 U.S.C. §119(e) to U.S. Provisional Application No. 62/058,557 filed Oct. 1, 2014, which application is hereby incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 191172_401WO_SEQUENCE LISTING.txt. The text file is 63.9 KB, was created on Sep. 30, 2015, and is being submitted electronically via EFS-Web.

### BACKGROUND

### Technical Field

Embodiments of the presently disclosed invention relate generally to virology and molecular pharmacology. In particular the present embodiments relate to antiviral polypeptides, compositions comprising such polypeptides, and methods of using the same. More specifically, the present embodiments relate to antiviral polypeptides of 15-50 amino acids, and in certain embodiments to antiviral polypeptides of 25-30 amino acids, that substantially impair one or more viral functions against cells to which they exhibit tropism, such as cell binding, cell membrane fusion, cell entry, intracellular viral replication and/or assembly, and lysis of infected cells.

### Description of the Related Art

Viruses and the host cells that they are capable of infecting (e.g., to which they exhibit tropism) exist in a constantly changing equilibrium as each adapts to evade detection and destruction by the other. Host organisms respond to viral infections by developing adaptive and innate immune responses and genetic resistance and/or by pharmacological intervention, while viruses adapt to the host's immune surveillance and antiviral drugs, for example by rapid genetic evolution by which drug resistance and/or antigenic modifications may be selected. Consequently, effective antiviral strategies require frequent replenishment to mitigate the fading efficacy of previously deployed pharmaceuticals.

Global climate change may be another factor that influences the virus-host cell equilibrium. Changing weather patterns alter bird migration patterns and other viral host ranges and expand the opportunities for exposure of viral vectors to new organisms, creating an environment in which can arise novel antigenic combinations that evade host immune surveillance. As tropical and subtropical viral vectors are expanding into new geographic regions, outbreaks of viral infections are remaining active longer and in wider geographic areas, thereby increasing the risk to human populations.

For example, the West Nile virus, a mosquito-borne flavivirus that infects birds and mammals, was first observed in the U.S. in New York City in 1999, and expanded rapidly westward across the country. By 2013, more than 39,500 cases and 1,668 deaths were recorded by the United States Centers for Disease Control (CDC). As another example, in December 2013 and well into 2014, a new strain of Ebola virus emerged in the West African nation of Guinea and spread to neighbouring Liberia and Sierra Leone (Baize, et al, 2014 New Eng. J. Med. DOI: 10.1056/NEJMoa1404505). Ebola disease outbreaks occur primarily in remote villages near the tropical rainforests of Gabon and the Republic of Congo and the disease had not previously been reported in Guinea. With a case fatality rate of 70.8%, the World Health Organization (WHO) recorded 4507 cases in five West African nations in this most serious outbreak since the discovery of the virus in 1976 (WHO Ebola Response Team, 2014 New Eng. J. Med. DOI: 10.1056/NEJMoa1411100). Since Ebola outbreaks typically follow the cessation of the rainy season, the risk of a geographic expansion of Ebola as a result of climate change had been predicted and discussed in numerous studies (Pinzon et al., 2004 Am. J. Trop. Med. Hyg. 71:664; Peterson et al., 2004 Emerg. Infect. Dis. 10:40; Tucker et al., 2002 Photogr. Engin. Remote Sens. 2:147).

Concurrently in December 2013, the mosquito-borne chikungunya virus was confirmed on the Caribbean island of St. Martin in the first documented transmissions in the Western Hemisphere of the disease to humans from infected mosquitoes. Endemic in sub Saharan Africa, the Philippines, Taiwan, and Australia, the chikungunya virus causes debilitating joint pain, fever and rash, and has no treatment or vaccine. The virus is typically transmitted by the tropical Aedes aegypti mosquito, but has adapted to the Asian tiger mosquito (A. albopictus) native to the southern U.S. (Tsetsarkin et al., 2007 PLoS Pathog. 3(12):e201). In June 2014 two mosquito-transmitted chikunguya cases were reported in Florida (Kuehn, 2014, JAMA. 2014; 312(8):776-777. doi:10.1001/jama.2014.9916) confirming the presence of the virus in the U.S.

Previously unknown viruses such as the coronavirus responsible for the Middle East Respiratory Syndrome (MERS) have emerged in Saudi Arabia, Jordan, Qatar, the United Arab Emirates, and the United States, infecting 636 resulting in 193 deaths (Global Alert Response, World Health Organization, May 28, 2014). Similar to the Severe Acute Respiratory Syndrome (SARS) epidemic in 2003 which killed more than 900 people and crippled hospital systems in China, Hong Kong, Vietnam, and Canada, a novel coronavirus jumped from an animal reservoir to an immune-naïve human population and is spreading through clinics and hospitals. Climate change may also have the potential to reintroduce the Variola virus, the causative agent of smallpox that was believed to be eradicated in 1979 after a global vaccination campaign by the World Health Organization, as a result of the rapid thawing of frozen corpses in the Siberian tundra that harbor the dormant virus (Stone, 2002 Science 295:5562).

Herpes Simplex Virus

Herpes simplex virus 1 (HSV1) infections are incurable, and once a subject has been infected the virus remains in the body for life. The primary site of HSV1 infections is the oral mucosa, with viral replication resulting in eruptions of mucosal ulcers. The related HSV2 virus is sexually transmitted and infects genital mucosa. Repeated outbreaks are common and can result from exposure to ultraviolet light, immune suppression, and trauma to the nerve ganglia, which harbor latent virus. The herpes virus can also infect the cornea, resulting in more than a half million cases per year of ocular keratitis; ocular herpes infection is the second leading cause of corneal blindness in the U.S.

The CDC estimates 29% and 9% of the U.S. population harbor HSV1 and HSV2, respectively (Centers for Disease Control and Prevention (CDC). National Center for Health Statistics (NCHS). National Health and Nutrition Examination Survey Data. Hyattsville, Md.: U.S. Department of Health and Human Services, Centers for Disease Control and Prevention, March 2010). Treatments to suppress recurrent outbreaks include the nucleoside analogs acyclovir, valacyclovir, famciclovir, and penciclovir. To be effective however, the medications must be administered daily, resulting in high treatment costs and the potentials for drug toxicity and induction of drug resistant virus strains. Therefore, there is a strong demand to identify novel anti-HSV molecules as candidate pharmaceuticals for inexpensive and safer alternatives to the nucleoside analogs.

Influenza Virus

Infection with the influenza virus results in fever, chills, nasal discharge, sore throat, muscle pains, severe headache, coughing, and fatigue. The virus is commonly transmitted by aerosols from sneezes and coughs but can also be transmitted after close contact with swine, for instance, in farm workers or in children visiting county fairs. Individuals with compromised immune systems, pregnant women and children are particularly susceptible to life-threatening complications of influenza infections, such as pneumonia. The World Health Organization estimates that globally, the influenza virus infects three to five million people annually, and causes 250,000 to 500,000 deaths. In pandemic years, infection rates can be ten times greater. Common antivirals used to treat influenza infections include neuraminidase (NA) inhibitors such oseltamivir and zanamivir, and the M2 protein inhibitors amantadine and rimantadine. The NA inhibitors reduce the duration of symptoms by less than 24 hours but do not reduce complications that result in hospitalization (Ebell et al., 2013 Fam. Practice 30(2):125). The M2 protein inhibitors block viral entry into the cell; however the widespread agricultural use and over-the-counter availability of M2 protein inhibitors in China and Russia has resulted in a high rate of viral resistance. The CDC estimates 91% of the H3N2 influenza strain is resistant and therefore recommends against using M2 inhibitors in 2005 due to the high rate of resistance (CDC, 2006 Morbid. Mortal. Wkly Rep. 55(2):44).

Clearly, in light of constant viral adaptations to environmental changes, vaccines and pharmaceuticals, and in view of the potential for viral epidemics to cause massive morbidity, mortality and economic disruption, there is a need for improved broad spectrum antiviral strategies, including formulation, production and timely distribution of antiviral agents. The rapid environmental changes associated with global climate change further escalate the immediate need for novel antiviral agents. The presently disclosed invention embodiments address these needs and provide other related advantages.

### BRIEF SUMMARY

In one aspect of the present invention, there is provided an antiviral polypeptide of at least 15, 16, 17, 18, 19, 20, 21, 22, 23, 24, or 25 amino acids and not more than 50, 49, 48, 47, 46, 45, 44, 43, 42, 41, 40, 39, 38, 37, 36, 35, 34, 33, 32, 31 or 30 amino acids, comprising a peptide of general formula I: N-X-C [I] wherein: (a) N is an amino terminus of the antiviral polypeptide and either (1) N consists of 0, 1, 2, 3, 4, 5, 6, 7, 8, 9, 10, 11, 12, 13, 14, 15, 16, 17, 18, 19, or 20 amino acids that are independently selected from natural and non-natural amino acids, or (2) N is an amino terminus of the antiviral polypeptide of general formula II: N1-N2 [II] wherein: N1 is a non-natural amino acid and N2 consists of 0, 1, 2, 3, 4, 5, 6, 7, 8, 9, 10, 11, 12, 13, 14, 15, 16, 17, 18, or 19 amino acids that are independently selected from natural and non-natural amino acids; (b) C is a carboxy terminus of the antiviral polypeptide and either (1) C consists of 0, 1, 2, 3, 4, 5, 6, 7, 8, 9, 10, 11, 12, 13, 14, 15, 16, 17, 18, 19, or 20 amino acids that are independently selected from natural and non-natural amino acids, or (2) C is a carboxy terminus of the antiviral polypeptide of general formula II: C1-C2 [II] wherein: C1 consists of 0, 1, 2, 3, 4, 5, 6, 7, 8, 9, 10, 11, 12, 13, 14, 15, 16, 17, 18, or 19 amino acids that are independently selected from natural and non-natural amino acids and C2 is a non-natural amino acid; and (c) X is a peptide of 30, 29, 28, 27, 26, 25, 24, 23, 22, 21, 20, 19, 18, 17, 16, or 15 amino acids and X is one of: (1) a peptide of general formula III: X1-X2-X3-X4-X5-X6-X7-X8-X9-X10-X11-X12-X13-X14-X15-X16-X17-X18-X19-X20-X21-X22-X23-X24-X25-X26-X27-X28-X29-X30 [III] [SEQ ID NO:156] wherein:

X1 is R, K, H, N, E, D, or Q;
X2 is Q, R, E, H, K, S, T, or C;
X3 is Y, H, F, or W;
X4 is S, A, N, T, C, or Q;
X5 is V, I, L, M, G, A, or L;
X6 is T, S, C, or Q;
X7 is D, N, E, K, or R;
X8 is G, A, V, L, M, I, or S;
X9 is L, I, M, F, V, G, or A;
X10 is E, D, Q, K, H, R, or N;
X11 is D, N, E, K, or R;
X12 is Y, H, F, or W;
X13 is N, D, H, S, K, R, or E;
X14 is T, S, C, or Q;
X15 is S, A, N, T, C, or Q;
X16 is P;
X17 is Q, R, E, H, K, S, T, or C;
X18 is S, A, N, T, C, or Q;
X19 is T, S, C, or Q;
X20 is E, D, Q, K, H, R, or N;
X21 is E, D, Q, K, H, R, or N;
X22 is V, I, L, M, G, A, or L;
X23 is V, I, L, M, G, A, or L;
X24 is Q, R, E, H, K, S, T, or C;
X25 is S, A, N, T, C, or Q;
X26 is F, L, W, or Y;
X27 is L, I, M, F, V, G, or A;
X28 is I, L, M, V, G, or A;
X29 is S, A, N, T, C, or Q;
X30 is Q, R, E, H, K, S, T, or C;

(2) a peptide of general formula IV: X1-X2-X3-X4-X5-X6-X7-X8-X9-X10-X11-X12-X13-X14-X15-X16-X17-X18-X19-X20-X21-X22-X23-X24-X25-X26-X27-X28-X29-X30 [IV] [SEQ ID NO:157] wherein

X1 is R, K, H, N, E, D, or Q;
X2 is Q, R, E, H, K, S, T, or C;
X3 is Y, H, F, or W;
X4 is S, A, N, T, C, or Q;
X5 is V, I, L, M, G, A, or L;
X6 is T, S, C, or Q;
X7 is D, N, E, K, or R;
X8 is G, A, V, L, M, I, or S;
X9 is L, I, M, F, V, G, or A;
X10 is E, D, Q, K, H, R, or N;
X11 is D, N, E, K, or R;
X12 is Y, H, F, or W;
X13 is S, A, N, T, C, or Q;
X14 is T, S, C, or Q;
X15 is S, A, N, T, C, or Q;
X16 is P;
X17 is Q, R, E, H, K, S, T, or C;
X18 is S, A, N, T, C, or Q;
X19 is T, S, C, or Q;
X20 is E, D, Q, K, H, R, or N;
X21 is E, D, Q, K, H, R, or N;
X22 is V, I, L, M, G, A, or L;
X23 is V, I, L, M, G, A, or L;
X24 is Q, R, E, H, K, S, T, or C;
X25 is S, A, N, T, C, or Q;
X26 is F, L, W, or Y;
X27 is L, I, M, F, V, G, or A;
X28 is I, L, M, V, G, or A;
X29 is S, A, N, T, C, or Q;
X30 is Q, R, E, H, K, S, T, or C;

(3) peptide of general formula V:

[V]
[SEQ ID NO: 158]
X1-X2-X3-X4-X5-X6-X7-X8-X9-X10-X11-X12-X13-X14-X15-
X16-X17-X18-X19-X20-X21-X22-X23-X24-X25-X26-X27-
X28-X29-X30

wherein

X1 is A, G, A, V, L, M, I, or S;
X2 is D, N, E, K, or R;
X3 is V, I, L, M, G, A, or L;
X4 is D, N, E, K, or R;
X5 is V, I, L, M, G, A, or L;
X6 is S, A, N, T, C, or Q;
X7 is A, G, A, V, L, M, I, or S;
X8 is V, I, L, M, G, A, or L;
X9 is Q, R, E, H, K, S, T, or C;
X10 is A, G, A, V, L, M, I, or S;
X11 is K, R, E, Q, H, N, or D;
X12 is L, I, M, F, V, G, or A;
X13 is G, A, V, L, M, or I;
X14 is A, G, A, V, L, M, I, or S;
X15 is L, I, M, F, V, G, or A;
X16 is E, D, Q, K, H, R, or N;
X17 is L, I, M, F, V, G, or A;
X18 is N, D, H, S, K, R, or E;
X19 is Q, R, E, H, K, S, T, or C;
X20 is R, K, H, N, E, D, or Q;
X21 is D, N, E, K, or R;
X22 is A, G, A, V, L, M, I, or S;
X23 is A, G, A, V, L, M, I, or S;
X24 is A, G, A, V, L, M, I, or S;
X25 is E, D, Q, K, H, R, or N;
X26 is T, S, C, or Q;
X27 is E, D, Q, K, H, R, or N;
X28 is L, I, M, F, V, G, or A;
X29 is R, K, H, N, E, D, or Q;
X30 is V, I, L, M, G, A, or L;

(4) a peptide of general formula VI:

[VI]
[SEQ ID NO: 159]
X1-X2-X3-X4-X5-X6-X7-X8-X9-X10-X11-X12-X13-X14-X15-
X16-X17-X18-X19-X20-X21-X22-X23-X24-X25

wherein:

X1 is G, A, V, L, M, or I;
X2 is D, N, E, K, or R;
X3 is T, S, C, or Q;
X4 is V, I, L, M, G, A, or L;
X5 is G, A, V, L, M, or I;
X6 is L, I, M, F, V, G, or A;
X7 is I, L, M, V, G, or A;
X8 is D, N, E, K, or R;
X9 is E, D, Q, K, H, R, or N;
X10 is Q, R, E, H, K, S, T, or C;
X11 is N, D, H, S, K, R, or E;
X12 is E, D, Q, K, H, R, or N;
X13 is A, G, A, V, L, M, I, or S;
X14 is S, A, N, T, C, or Q;
X15 is K, R, E, Q, H, N, or D;
X16 is T, S, C, or Q;
X17 is N, D, H, S, K, R, or E;
X18 is G, A, V, L, M, or I;
X19 is L, I, M, F, V, G, or A;
X20 is G, A, V, L, M, or I;
X21 is A, G, A, V, L, M, I, or S;
X22 is A, G, A, V, L, M, I, or S;
X23 is E, D, Q, K, H, R, or N;
X24 is A, G, A, V, L, M, I, or S;
X25 is F, L, W, or Y;

(5) a peptide that comprises the amino acid sequence set forth in any one of SEQ ID NOS:1-155, (6) a peptide that comprises 15, 16, 17, 18, 19, 20, 21, 22, 23, 24, 25, 26, 27, 28, 29, or 30 contiguous amino acids of the amino acid sequence set forth in any one of SEQ ID NOS: 1-109, or (7) a peptide that comprises 15, 16, 17, 18, 19, 20, 21, 22, 23, 24, or 25 contiguous amino acids of the amino acid sequence set forth in any one of SEQ ID NOS:110-155.

In certain embodiments the antiviral polypeptide is capable of at least one antiviral activity that is selected from (i) substantially impairing binding of a virus to a cell to which the virus exhibits tropism; (ii) substantially impairing fusion of a virus to a cell membrane of a cell to which the virus exhibits tropism; (iii) substantially impairing viral entry by a virus into a cell to which the virus exhibits tropism; (iv) substantially impairing viral replication or viral assembly by a virus in a cell to which the virus exhibits tropism; (v) substantially impairing release from a virus-infected cell of viral particles that have been synthesized in the cell as a result of infection by the virus; and (vi) substantially impairing lysis of a virus-infected cell that results from infection of the cell by the virus.

In another embodiment there is provided a fusion protein which comprises the antiviral polypeptide described above. In another embodiment there is provided the antiviral polypeptide described above in which at least one amino acid situated at an identified amino acid sequence position in the amino acid sequence of the polypeptide comprises at least one of (i) a non-naturally occurring amino acid, or (ii) an amino acid that is not found at the identified amino acid sequence position in any naturally occurring homologue having at least 90% sequence identity to the antiviral polypeptide. In another embodiment there is provided a pharmaceutical composition comprising the antiviral polypeptide described above; and a pharmaceutical carrier or excipient.

Turning to another embodiment, there is provided a method of substantially impairing a viral activity in a cell, comprising contacting the cell with the antiviral polypeptide of any one of claim 1-4, wherein the viral activity that is substantially impaired comprises at least one of: (i) binding of a virus to a cell to which the virus exhibits tropism; (ii) fusion of a virus to a cell membrane of a cell to which the virus exhibits tropism; (iii) viral entry by a virus into a cell to which the virus exhibits tropism; (iv) viral replication or viral assembly by a virus in a cell to which the virus exhibits tropism; (v) release from a virus-infected cell of viral particles that have been synthesized in the cell as a result of infection by the virus; and (vi) lysis of a virus-infected cell that results from infection of the cell by the virus. In certain further embodiments the cell is contacted with the antiviral polypeptide in vitro.

In another embodiment there is provided a method of reducing likelihood or severity of viral infection in a subject, comprising administering to the subject a therapeutically effective amount of the pharmaceutical composition described above. In certain other embodiments there is provided a method for treating a subject having or suspected of being at risk for having a viral infection, comprising administering to the subject a therapeutically effective amount of the pharmaceutical composition described above.

These and other aspects of the invention will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet, are incorporated herein by reference in their entirety, as if each was incorporated individually. Aspects of the invention can be modified, if necessary, to employ concepts of the various patents, applications and publications to provide yet further embodiments of the invention.

### BRIEF DESCRIPTION OF THE SEVERAL VIEWS OF THE DRAWINGS

FIG. 1 shows inhibition by antiviral peptides [SEQ ID NOS:53-54] described herein in an in vitro plaque assay of herpes simplex virus (HSV1 strain KOS) replication in Vero cells.

FIG. 2 shows inhibition by antiviral peptides [SEQ ID NOS:53-54] described herein in an in vitro plaque assay of herpes simplex virus (HSV1 strain KOS) replication in Vero cells.

FIG. 3 shows inhibition by antiviral peptides [SEQ ID NOS:109 and 155] described herein in an in vitro plaque assay of influenza virus (H3N2/Wisconsin/67/2005) replication in MDCK cells.

FIG. 4 shows inhibition by antiviral peptides [SEQ ID NOS:109 and 155] described herein in an in vitro plaque assay of influenza virus (H3N2/Wisconsin/67/2005) replication in MDCK cells.

### DETAILED DESCRIPTION

The present disclosure relates to antiviral polypeptides and peptides as described herein, including variants as also described herein, having broad antiviral activity which may be manifest as one or more of the abilities to:
(i) substantially impair binding of a virus to a cell to which the virus exhibits tropism; (ii) substantially impair fusion of a virus to a cell membrane of a cell to which the virus exhibits tropism; (iii) substantially impair viral entry by a virus into a cell to which the virus exhibits tropism; (iv) substantially impair viral replication or viral assembly by a virus in a cell to which the virus exhibits tropism; (v) substantially impair release from a virus-infected cell of viral particles that have been synthesized in the cell as a result of infection by the virus; and (vi) substantially impair lysis of a virus-infected cell that results from infection of the cell by the virus.

Certain embodiments will thus usefully exploit the antiviral properties of the herein disclosed antiviral polypeptides and peptides in compositions and methods wherein any of a wide range of such antiviral activity may be desired, including in pharmaceutical compositions. Among certain preferred embodiments, substantial impairment of viral activity in a cell is contemplated in vivo and/or in vitro following a step of contacting the herein described antiviral polypeptide with the cell or the virus or both the cell and the virus, for example, in a method of substantially impairing a viral activity in a cell in vitro, or in a method of reducing likelihood or severity of viral infection in a subject, or in a method for treating a subject having or suspected of being at risk for having a viral infection.

The presently described broadly antiviral peptides were surprisingly identified as sequence fragments in a genomic screen for the signatures of human genes in geographically defined human populations that have survived by adaptation to centuries of endemic viral infections. The survival proteins encoded by so-identified viral resistance genes are believed according to non-limiting theory to represent components of a common viral trafficking pathway that has apparently been exploited by multiple viruses including viruses other than those responsible for earlier selective pressures. Exemplified here are peptides that are presently shown to inhibit proliferation of genetically disparate viruses such as herpes simplex virus 1 and influenza A H3N2. In view of the common cellular trafficking pathway components that may be exploited by a broad range of genetically dissimilar viruses, the presently disclosed peptides are similarly contemplated as having antiviral activity against a wide range of viruses that are human pathogens and also against a wide range of viruses that are pathogens in non-human animals and also against a wide range of viruses that are pathogens in plants.

The use of peptides as anti-viral pharmaceuticals also provides advantages over current therapies for treating viral infections. Peptides are relatively inexpensive to synthesize and can be designed to interrupt multiple stages of the viral replication cycle such as cell receptor binding, cell membrane fusion, endocytosis or invasion, ingress, replication, viral gene expression, viral genome packaging, assembly of infectious virions, viral egress from infected cells, and host cell lysis. For example, specific peptides have been designed to adhere to the HSV1 enzyme ribonucleotide reductase and disrupt the binding of the enzyme subunits, or interfere with viral proteinase. Additionally, by targeting multiple stages of the viral replication cycle, opportunities for the virus to adapt and develop resistance are mitigated.

Accordingly, in certain embodiments the present disclosure provides an antiviral polypeptide of at least 15, 16, 17, 18, 19, 20, 21, 22, 23, 24, or 25 amino acids and not more than 50, 49, 48, 47, 46, 45, 44, 43, 42, 41, 40, 39, 38, 37, 36, 35, 34, 33, 32, 31 or 30 amino acids, comprising a peptide of general formula I:

N-X-C [I] wherein:

(a) N is an amino terminus of the antiviral polypeptide and either
(1) N consists of 0, 1, 2, 3, 4, 5, 6, 7, 8, 9, 10, 11, 12, 13, 14, 15, 16, 17, 18, 19, or 20 amino acids that are independently selected from natural and non-natural amino acids, or
(2) N is an amino terminus of the antiviral polypeptide of general formula II:

N1-N2 [II] wherein:

N1 is a non-natural amino acid and N2 consists of 0, 1, 2, 3, 4, 5, 6, 7, 8, 9, 10, 11, 12, 13, 14, 15, 16, 17, 18, or 19 amino acids that are independently selected from natural and non-natural amino acids;

(b) C is a carboxy terminus of the antiviral polypeptide and either
(1) C consists of 0, 1, 2, 3, 4, 5, 6, 7, 8, 9, 10, 11, 12, 13, 14, 15, 16, 17, 18, 19, or 20 amino acids that are independently selected from natural and non-natural amino acids, or
(2) C is a carboxy terminus of the antiviral polypeptide of general formula II:

C1-C2 [II] wherein:

C1 consists of 0, 1, 2, 3, 4, 5, 6, 7, 8, 9, 10, 11, 12, 13, 14, 15, 16, 17, 18, or 19 amino acids that are independently selected from natural and non-natural amino acids and C2 is a non-natural amino acid; and

(c) X is a peptide of 30, 29, 28, 27, 26, 25, 24, 23, 22, 21, 20, 19, 18, 17, 16, or 15 amino acids and X is one of:
(1) a peptide of general formula III: X1-X2-X3-X4-X5-X6-X7-X8-X9-X10-X11-X12-X13-X14-X15-X16-X17-X18-X19-X20-X21-X22-X23-X24-X25-X26-X27-X28-X29-X30 [III] [SEQ ID NO:156]

wherein:

X1 is R, K, H, N, E, D, or Q;
X2 is Q, R, E, H, K, S, T, or C;
X3 is Y, H, F, or W;
X4 is S, A, N, T, C, or Q;
X5 is V, I, L, M, G, A, or L;
X6 is T, S, C, or Q;
X7 is D, N, E, K, or R;
X8 is G, A, V, L, M, I, or S;
X9 is L, I, M, F, V, G, or A;
X10 is E, D, Q, K, H, R, or N;
X11 is D, N, E, K, or R;
X12 is Y, H, F, or W;
X13 is N, D, H, S, K, R, or E;
X14 is T, S, C, or Q;
X15 is S, A, N, T, C, or Q;
X16 is P;
X17 is Q, R, E, H, K, S, T, or C;
X18 is S, A, N, T, C, or Q;
X19 is T, S, C, or Q;
X20 is E, D, Q, K, H, R, or N;
X21 is E, D, Q, K, H, R, or N;
X22 is V, I, L, M, G, A, or L;
X23 is V, I, L, M, G, A, or L;
X24 is Q, R, E, H, K, S, T, or C;
X25 is S, A, N, T, C, or Q;
X26 is F, L, W, or Y;
X27 is L, I, M, F, V, G, or A;
X28 is I, L, M, V, G, or A;
X29 is S, A, N, T, C, or Q;
X30 is Q, R, E, H, K, S, T, or C;

(2) a peptide of general formula IV:

[IV]
[SEQ ID NO: 157]
X1-X2-X3-X4-X5-X6-X7-X8-X9-X10-X11-X12-X13-X14-
X15-X16-X17-X18-X19-X20-X21-X22-X23-X24-X25-X26-
X27-X28-X29-X30

wherein

X1 is R, K, H, N, E, D, or Q;
X2 is Q, R, E, H, K, S, T, or C;
X3 is Y, H, F, or W;
X4 is S, A, N, T, C, or Q;
X5 is V, I, L, M, G, A, or L;
X6 is T, S, C, or Q;
X7 is D, N, E, K, or R;
X8 is G, A, V, L, M, I, or S;
X9 is L, I, M, F, V, G, or A;
X10 is E, D, Q, K, H, R, or N;
X11 is D, N, E, K, or R;
X12 is Y, H, F, or W;
X13 is S, A, N, T, C, or Q;
X14 is T, S, C, or Q;
X15 is S, A, N, T, C, or Q;
X16 is P;
X17 is Q, R, E, H, K, S, T, or C;
X18 is S, A, N, T, C, or Q;
X19 is T, S, C, or Q;
X20 is E, D, Q, K, H, R, or N;
X21 is E, D, Q, K, H, R, or N;
X22 is V, I, L, M, G, A, or L;
X23 is V, I, L, M, G, A, or L;
X24 is Q, R, E, H, K, S, T, or C;
X25 is S, A, N, T, C, or Q;
X26 is F, L, W, or Y;
X27 is L, I, M, F, V, G, or A;
X28 is I, L, M, V, G, or A;
X29 is S, A, N, T, C, or Q;
X30 is Q, R, E, H, K, S, T, or C;

(3) a peptide of general formula V:

[V]
[SEQ ID NO: 158]
X1-X2-X3-X4-X5-X6-X7-X8-X9-X10-X11-X12-X13-X14-X15-
X16-X17-X18-X19-X20-X21-X22-X23-X24-X25-X26-X27-
X28-X29-X30

wherein

X1 is A, G, A, V, L, M, I, or S;
X2 is D, N, E, K, or R;
X3 is V, I, L, M, G, A, or L;
X4 is D, N, E, K, or R;
X5 is V, I, L, M, G, A, or L;
X6 is S, A, N, T, C, or Q;
X7 is A, G, A, V, L, M, I, or S;
X8 is V, I, L, M, G, A, or L;
X9 is Q, R, E, H, K, S, T, or C;
X10 is A, G, A, V, L, M, I, or S;
X11 is K, R, E, Q, H, N, or D;
X12 is L, I, M, F, V, G, or A;
X13 is G, A, V, L, M, or I;
X14 is A, G, A, V, L, M, I, or S;
X15 is L, I, M, F, V, G, or A;
X16 is E, D, Q, K, H, R, or N;
X17 is L, I, M, F, V, G, or A;
X18 is N, D, H, S, K, R, or E;
X19 is Q, R, E, H, K, S, T, or C;
X20 is R, K, H, N, E, D, or Q;
X21 is D, N, E, K, or R;
X22 is A, G, A, V, L, M, I, or S;
X23 is A, G, A, V, L, M, I, or S;
X24 is A, G, A, V, L, M, I, or S;
X25 is E, D, Q, K, H, R, or N;
X26 is T, S, C, or Q;
X27 is E, D, Q, K, H, R, or N;
X28 is L, I, M, F, V, G, or A;
X29 is R, K, H, N, E, D, or Q;
X30 is V, I, L, M, G, A, or L;

(4) a peptide of general formula VI:

[VI]
[SEQ ID NO: 159]
X1-X2-X3-X4-X5-X6-X7-X8-X9-X10-X11-X12-X13-X14-X15-
X16-X17-X18-X19-X20-X21-X22-X23-X24-X25

wherein:

X1 is G, A, V, L, M, or I;
X2 is D, N, E, K, or R;
X3 is T, S, C, or Q;
X4 is V, I, L, M, G, A, or L;
X5 is G, A, V, L, M, or I;
X6 is L, I, M, F, V, G, or A;
X7 is I, L, M, V, G, or A;
X8 is D, N, E, K, or R;
X9 is E, D, Q, K, H, R, or N;
X10 is Q, R, E, H, K, S, T, or C;
X11 is N, D, H, S, K, R, or E;
X12 is E, D, Q, K, H, R, or N;
X13 is A, G, A, V, L, M, I, or S;
X14 is S, A, N, T, C, or Q;
X15 is K, R, E, Q, H, N, or D;
X16 is T, S, C, or Q;
X17 is N, D, H, S, K, R, or E;
X18 is G, A, V, L, M, or I;
X19 is L, I, M, F, V, G, or A;
X20 is G, A, V, L, M, or I;
X21 is A, G, A, V, L, M, I, or S;
X22 is A, G, A, V, L, M, I, or S;
X23 is E, D, Q, K, H, R, or N;
X24 is A, G, A, V, L, M, I, or S;
X25 is F, L, W, or Y;

(5) a peptide that comprises the amino acid sequence set forth in any one of SEQ ID NOS:1-155,
(6) a peptide that comprises 15, 16, 17, 18, 19, 20, 21, 22, 23, 24, 25, 26, 27, 28, 29, or 30 contiguous amino acids of the amino acid sequence set forth in any one of SEQ ID NOS: 1-109, or
(7) a peptide that comprises 15, 16, 17, 18, 19, 20, 21, 22, 23, 24, or 25 contiguous amino acids of the amino acid sequence set forth in any one of SEQ ID NOS:110-155.

In preferred embodiments the antiviral polypeptide is capable of at least one antiviral activity that is selected from (i) substantially impairing binding of a virus to a cell to which the virus exhibits tropism; (ii) substantially impairing fusion of a virus to a cell membrane of a cell to which the virus exhibits tropism; (iii) substantially impairing viral entry by a virus into a cell to which the virus exhibits tropism; (iv) substantially impairing viral replication or viral assembly by a virus in a cell to which the virus exhibits tropism; (v) substantially impairing release from a virus-infected cell of viral particles that have been synthesized in the cell as a result of infection by the virus; and (vi) substantially impairing lysis of a virus-infected cell that results from infection of the cell by the virus. An antiviral activity that is substantially impaired refers to substantial and statistically significant, but not necessarily complete, inhibition of the indicated viral function, e.g., at least 50%, 60%, 70%, 80%, 85%, 90%, 95% or greater inhibition relative to appropriate untreated controls, according to art-accepted criteria for determining the indicated viral function. Quantitative functional assays for assessing viral activity are described in laboratory manuals such as Virology Methods Manual, Brian Mahy and Hillar Kangro, eds. Academic Press, 1996. ISBN: 978-0-12-465330-6.

### Polypeptides and Proteins

The terms “polypeptide” “protein” and “peptide” are used interchangeably and mean a polymer of amino acids not limited to any particular length. The term does not exclude modifications such as myristylation, sulfation, glycosylation, phosphorylation and addition or deletion of signal sequences. The term “polypeptide” or “protein” means one or more chains of amino acids, wherein each chain comprises amino acids covalently linked by peptide bonds, and wherein the polypeptide or protein may comprise one chain in certain preferred embodiments but may in other embodiments comprise a plurality of chains non-covalently and/or covalently linked together by peptide bonds. The present antiviral polypeptides may be produced wholly by synthetic chemistry or may be produced by non-naturally occurring, genetically-engineered or recombinant cells, and may comprise molecules having the amino acid sequences of generic formulae I-IV [SEQ ID NOS:156-159] as disclosed herein, or any of the amino acid sequences set forth in SEQ ID NOS:1-155. Thus, a “polypeptide” or a “protein” can comprise one (termed “a monomer”) or a plurality (termed “a multimer”) of amino acid chains. The terms “peptide,” “polypeptide” and “protein” specifically encompass the antiviral polypeptides of the present disclosure, or sequences that have deletions from, additions to, and/or substitutions of one or more amino acid of an antiviral polypeptide.

In preferred embodiments, the herein described antiviral peptides, such as any of the presently disclosed polypeptides having an amino acid sequence set forth in one of SEQ ID NOS:1-159, may be chemically modified by either or both of amidation at the amino terminus or acetylation at the carboxy terminus, which chemical modifications give rise to artificial peptides having chemical structures that do not occur naturally.

The terms “isolated protein” and “isolated polypeptide” referred to herein means that a subject protein or polypeptide (1) is not associated (by covalent or noncovalent interaction) with portions of a protein or polypeptide with which the “isolated protein” or “isolated polypeptide” may be associated in nature, (2) is operably associated (by covalent or noncovalent interaction) with a polypeptide with which it is not associated in nature, or (3) does not occur in nature. Such an isolated protein or polypeptide can be encoded by genomic DNA, cDNA, mRNA or other RNA, of may be of synthetic origin according to any of a number of well known chemistries for artificial peptide and protein synthesis, or any combination thereof.

Certain preferred embodiments contemplate wholly artificial chemical synthesis of the herein described antiviral peptides or polypeptides according to any of a number of established methodologies, such as those described in Amino Acid and Peptide Synthesis (Jones, J., 2002 Oxford Univ. Press USA, New York), Ramakers et al. (2014 Chem. Soc. Rev. 43:2743), Verzele et al. (2013 Chembiochem. 14:1032), Chandrudu et al. (2013 Molecules 18:4373), and/or Mäde et al. (2004 Beilstein J. Org. Chem. 10:1197). For example, manual or preferably automated solid-phase peptide synthesis based on the Merrifield method or other solid-phase peptide synthetic techniques and subsequent improvements (e.g., Merrifield, 1963 J. Am. Chem. Soc. 85:2149; Mitchell et al., 1978 J. Org. Chem. 43:2485; Albericio, F. (2000). Solid-Phase Synthesis: A Practical Guide (1 ed.). Boca Raton: CRC Press; Nilsson et al., 2005 Annu. Rev. Biophys. Biomol. Struct. 34; Schnolzer et al., Int. J. Peptide Res. Therap. 13 (1-2): 31; Li et al. 2013 Molecules 18:9797) are routine in the peptide synthesis art and may be employed to chemically synthesize the herein described antiviral polypeptides.

The term “polypeptide fragment” refers to a polypeptide, which can be monomeric or multimeric, that has an amino-terminal deletion, a carboxyl-terminal deletion, and/or an internal deletion or substitution of a naturally-occurring or recombinantly-produced polypeptide. As used herein, “contiguous amino acids” refers to covalently linked amino acids corresponding to an uninterrupted linear portion of a disclosed amino acid sequence. In certain embodiments, a polypeptide fragment can comprise an amino acid chain at least 5 to about 50 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids long.

Polypeptides may comprise a signal (or leader) sequence at the N-terminal end of the protein, which co-translationally or post-translationally directs transfer of the protein. The polypeptide may also be fused in-frame or conjugated to a linker or other sequence for ease of synthesis, purification or identification of the polypeptide (e.g., poly-His), or to enhance binding of the polypeptide to a solid support. Fusion domain polypeptides may be joined to the polypeptide at the N-terminus and/or at the C-terminus, and may include as non-limiting examples, immunoglobulin-derived sequences such as Ig constant region sequences or portions thereof, affinity tags such as His tag (e.g., hexahistidine or other polyhistidine), FLAG™ or myc or other peptide affinity tags, detectable polypeptide moieties such as green fluorescent protein (GFP) or variants thereof (e.g., yellow fluorescent protein (YFP), blue fluorescent protein (BFP), other aequorins or derivatives thereof, etc.) or other detectable polypeptide fusion domains, enzymes or portions thereof such as glutathione-S-transferase (GST) or other known enzymatic detection and/or reporter fusion domains, and the like, as will be familiar to the skilled artisan.

Cysteine-containing peptides may be used as fusion peptides that can be joined to the N- and/or C-terminus of the herein described antiviral polypeptides (e.g., SEQ ID NOS:1-155) to permit ready assembly of such polypeptides into disulfide-crosslinked dimers, trimers, tetramers or higher multimers according to established methodologies. For example, fusion polypeptides containing immunoglobulin gene superfamily member-derived sequences that include cysteine residues capable of forming interchain disulfide bridges are well known, as also are other strategies for engineering S-S linked multimers (e.g., Reiter et al., 1994 Prot. Eng. 7:697; Zhu et al., 1997 Prot. Sci. 6:781; Mabry et al., 2010 Mabs 2:20; Gao et al., 1999 Proc. Nat. Acad. Sci. USA 96:6025; Lim et al., 2010 Biotechnol. Bioeng. 106:27) Alternative approaches are also contemplated for grafting peptide sequences that promote multimer assembly as fusion domains onto a desired polypeptide such as the herein described antiviral peptides (e.g., Fan et al., 2008 FASEB J. 22:3795).

Polypeptide modifications may be effected biosynthetically and/or chemically according to a wide variety of well known methodologies. The presently disclosed antiviral peptides may have reactive molecules attached to their amino- and/or carboxy-terminal amino acid residues. These molecules may serve to tag the peptide and are useful in the synthesis, purification, and/or detection of the peptides, or to enhance solubility or cellular transit of the synthetic peptides. By way of illustration only, such tags may include biotin, streptavidin, FLAG, glutathione-S-transferase or calmodulin-binding peptide, for example, or any other tags well known in the art. The manipulation of peptides is described in laboratory manuals such as Molecular Cloning: A Laboratory Manual (2nd Ed.) by J. Sambrook, E. F. Fritsch, T. Maniatis, 1989, ISBN-13:

978-0879693091 ISBN-10: 0879693096; and Molecular Cloning: A Laboratory Manual (4th Ed.) by M. R. Green and J. Sambrook, 2012 Cold Spring Harbor Laboratory Press; ISBN-13: 978-1936113415ISBN-10: 1936113414. The term ‘amino acid’ means a compound which is incorporated in a naturally occurring polypeptide as either the L or D enantiomer. Such amino acids are described in biochemistry textbooks for example, Lehninger's Principles of Biochemistry (4th Ed.) by Nelson, D., and Cox, M.; W.H. Freeman and Company, New York, 2005, ISBN 0-7167-4339-6 and include alanine, arginine, asparagine, aspartic acid, cysteine, glutamic acid, glutamine, glycine, histidine, hydroxylysine, hydroxyproline, isoleucine, leucine, lysine, methionine, phenylalanine, proline, pyroglutamic acid, sarcosine, serine, threonine, tryptophan, tyrosine and valine.

In general, the abbreviations used herein represent the amino acid residues based on the terminology of the IUPAC-IUB Commission on Biochemical Nomenclature (see Biochemistry, 11, 1726-1732 (1972)). For instance the following single and three letter abbreviations are used to represent the following amino acids: G, Glycine (Gly); P, Proline (Pro); A, Alanine (Ala); V, Valine (Val); L, Leucine (Leu); I, Isoleucine (Ile); M, Methionine (Met); C, Cysteine (Cys); F, Phenylalanine (Phe); Y, Tyrosine (Tyr); W, Tryptophan (Trp); H, Histidine (His); K, Lysine (Lys); R, Arginine (Arg); Q, Glutamine (Gln); N, Asparagine (Asn); E, Glutamic Acid (Glu); D,

Aspartic Acid (Asp); S, Serine (Ser); and T, Threonine (Thr).

“Natural or non-natural amino acid” includes any of the common naturally occurring amino acids which serve as building blocks for the biosynthesis of peptides, polypeptides and proteins (e.g., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine(Y)) and also includes modified, derivatized, enantiomeric, rare and/or unusual amino acids, whether naturally occurring or synthetic, for instance, N-formylmethionine, hydroxyproline, hydroxylysine, desmosine, isodesmosine, ε-N-methyllysine, ε-N-trimethyllysine, methylhistidine, dehydrobutyrine, dehydroalanine, α-aminobutyric acid, β-alanine, γ-aminobutyric acid, homocysteine, homoserine, citrulline, ornithine and other amino acids that may be isolated from a natural source and/or that may be chemically synthesized, for instance, as may be found in Proteins, Peptides and Amino Acids Sourcebook (White, J. S. and White, D. C., 2002 Humana Press, Totowa, N.J.) or in Amino Acid and Peptide Synthesis (Jones, J., 2002 Oxford Univ. Press USA, New York) or in Unnatural Amino Acids, ChemFiles Vol. 1, No. 5 (2001 Fluka Chemie GmbH; Sigma-Aldrich, St. Louis, Mo.) or in Unnatural Amino Acids II, ChemFiles Vol. 2, No. 4 (2002 Fluka Chemie GmbH; Sigma-Aldrich, St. Louis, Mo.). Additional descriptions of natural and/or non-natural amino acids may be found, for example, in Kotha, 2003 Acc. Chem. Res. 36:342; Maruoka et al., 2004 Proc. Nat. Acad. Sci. USA 101:5824; Lundquist et al., 2001 Org. Lett. 3:781; Tang et al., 2002 J. Org. Chem. 67:7819; Rothman et al., 2003 J. Org. Chem. 68:6795; Krebs et al., 2004 Chemistry 10:544; Goodman et al., 2001 Biopolymers 60:229; Sabat et al., 2000 Org. Lett. 2:1089; Fu et al., 2001 J. Org. Chem. 66:7118; and Hruby et al., 1994 Meths. Mol. Biol. 35:249. The standard three-letter abbreviations and one-letter symbols are used herein to designate natural and non-natural amino acids.

Other non-natural amino acids or amino acid analogues are known in the art and include, but are not limited to, non-natural L or D derivatives (such as D-amino acids present in peptides and/or peptidomimetics such as those presented above and elsewhere herein), fluorescent labeled amino acids, as well as specific examples including O-methyl-L-tyrosine, an L-3-(2-naphthyl)alanine, a 3-methyl-phenylalanine, 3-idio-tyrosine, O-propargyl-tyrosine, homoglutamine, an O-4-allyl-L-tyrosine, a 4-propyl-L-tyrosine, a 3-nitro-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-acetyl-L-phenylalanine, an m-acetyl-L-phenylalanine, selenomethionine, telluromethionine, selenocysteine, an alkyne phenylalanine, an O-allyl-L-tyrosine, an O-(2-propynyl)-L-tyrosine, a p-ethylthiocarbonyl-L-phenylalanine, a p-(3-oxobutanoyl)-L-phenylalanine, a p-benzoyl-L-phenylalanine, an L-phosphoserine, a phosphonoserine, a phosphonotyrosine, homoproparglyglycine, azidohomoalanine, a p-iodo-phenylalanine, a p-bromo-L-phenylalanine, dihydroxy-phenylalanine, dihydroxyl-L-phenylalanine, a p-nitro-L-phenylalanine, an m-methoxy-L-phenylalanine, a p-iodo-phenylalanine, a p-bromophenylalanine, a p-amino-L-phenylalanine, and an isopropyl-L-phenylalanine, trifluoroleucine, norleucine (“Nle”), D-norleucine (“dNle” or “D-Nle”), 5-fluoro-tryptophan, para-halo-phenylalanine, homo-phenylalanine (“homo-Phe”), seleno-methionine, ethionine, S-nitroso-homocysteine, thia-proline, 3-thienyl-alanine, homo-allyl-glycine, trifluoroisoleucine, trans and cis-2-amino-4-hexenoic acid, 2-butynyl-glycine, allyl-glycine, para-azido-phenylalanine, para-cyano-phenylalanine, para-ethynyl-phenylalanine, hexafluoroleucine, 1,2,4-triazole-3-alanine, 2-fluoro-histidine, L-methyl histidine, 3-methyl-L-histidine, β-2-thienyl-L-alanine, β-(2-thiazolyl)-DL-alanine, homoproparglyglycine (HPG) and azidohomoalanine (AHA) and the like.

In certain embodiments a natural or non-natural amino acid may be present that comprises a hydrophobic side chain as found, for example, in alanine, valine, isoleucine, leucine, proline, phenylalanine, tryptophan or methionine or analogues thereof including in other natural or non-natural amino acids based on the structures of which the skilled person will readily recognize when a hydrophobic side chain (e.g., typically one that is non-polar when in a physiological milieu) is present. In certain embodiments a natural or non-natural amino acid may be present that comprises a basic side chain as found, for example, in lysine, arginine or histidine or analogues thereof including in other natural or non-natural amino acids based on the structures of which the skilled person will readily recognize when a basic (e.g., typically polar and having a positive charge when in a physiological milieu) is present. In certain embodiments a natural or non-natural amino acid may be present that comprises an acidic side chain as found, for example, in aspartic acid or glutamic acid or analogues thereof including in other natural or non-natural amino acids based on the structures of which the skilled person will readily recognize when an acidic (e.g., typically polar and having a negative charge when in a physiological milieu) is present.

Peptides disclosed herein may in certain embodiments include L- and/or D-amino acids so long as the biological activity of the peptide is maintained (e.g., antiviral activity). The antiviral peptides also may comprise in certain embodiments any of a variety of known artificial post-synthetic or post-translational covalent chemical modifications by reactions that may include chemical modification of N- and/or C-termini to block one or more reactive groups and/or to remove one or more charged moieties according to any of a number of standard methodologies. Additional post-synthetic or post-translational covalent modification of the herein described antiviral polypeptides may include glycosylation (e.g., N-linked oligosaccharide addition at asparagine residues, O-linked oligosaccharide addition at serine or threonine residues, glycation, or the like), fatty acylation, acetylation, formylation, PAGylation, PEGylation, and phosphorylation. Peptides herein disclosed may further include analogs, alleles and allelic variants which may contain amino acid deletions, or additions or substitutions of one or more amino acid residues with other naturally occurring amino acid residues or non-natural amino acid residues.

Peptide and non-peptide analogs may be referred to as peptide mimetics or peptidomimetics, and are known in the pharmaceutical industry (Fauchere, J. Adv. Drug Res. 15:29 (1986); Evans et al. J. Med. Chem. 30: 1229 (1987)). These compounds may contain one or more non-natural amino acid residue(s), one or more chemical modification moieties (for example, glycosylation, pegylation, fluorescence, radioactivity, or other moiety), and/or one or more non-natural peptide bond(s) (for example, a reduced peptide bond: —CH₂—NH₂—). Peptidomimetics may be developed by a variety of methods, including by computerized molecular modeling, random or site-directed mutagenesis, PCR-based strategies, chemical mutagenesis, and others.

As also described above, certain embodiments also relate to peptidomimetics, or “artificial” polypeptides. Such polypeptides may contain one or more amino acid insertions, deletions or substitutions, one or more altered or artificial peptide bond, one or more chemical moiety (such as polyethylene glycol, glycosylation, label, toxin, or other moiety), and/or one or more non-natural amino acid. Synthesis of peptidomimetics is well known in the art and may include altering proteins or polypeptides by chemical mutagenesis, single or multi-site-directed mutagenesis, PCR shuffling, use of altered aminoacyl tRNA or aminoacyl tRNA synthetase molecules, the use of “stop” codons such as amber suppressors, use of four or five base-pair codons, or other means.

Polypeptide modifications thus may also include conjugation to carrier proteins (e.g., keyhole limpet hemocyanin (KLH), bovine serum albumin (BSA), ovalbumin (OVA) or other molecules), and covalent or non-covalent immobilization on solid supports. Chemical or biosynthetic conjugation to a carrier is contemplated, according to certain embodiments, for generation of conjugates that are multivalent with respect to the herein described antiviral peptides. Also contemplated is detectable labeling of the present antiviral polypeptides with detectable indicator moieties (sometimes referred to as reporter moieties) such as fluorophores (e.g., FITC, TRITC, Texas Red, etc.). Examples of a broad range of detectable indicators (including colorimetric indicators) that may be selected for specific purposes are described in Haugland, 2002 Handbook of Fluorescent Probes and Research Products-Ninth Ed., Molecular Probes, Eugene, Oreg.; in Mohr, 1999 J. Mater. Chem., 9: 2259-2264; in Suslick et al., 2004 Tetrahedron 60:11133-11138; and in U.S. Pat. No. 6,323,039. (See also, e.g., Fluka Laboratory Products Catalog, 2001 Fluka, Milwaukee, Wis.; and Sigma Life Sciences Research Catalog, 2000, Sigma, St. Louis, Mo.) A detectable indicator may be a fluorescent indicator, a luminescent indicator, a phosphorescent indicator, a radiometric indicator, a dye, an enzyme, a substrate of an enzyme, an energy transfer molecule, or an affinity label.

Other detectable indicators for use in certain embodiments contemplated herein include affinity reagents such as antibodies, lectins, immunoglobulin Fc receptor proteins (e.g., Staphylococcus aureus protein A, protein G or other Fc receptors), avidin, biotin, other ligands, receptors or counterreceptors or their analogues or mimetics, and the like. For such affinity methodologies, reagents for immunometric measurements, such as suitably labeled antibodies or lectins, may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., 1987 Methods in Enzymology 135:30-65; Harlow and Lane, Antibodies: A Laboratory Manual, 1988 Cold Spring Harbor Laboratory, Cold Spring Harbor, N.Y.; Haugland, Handbook of Fluorescent Probes and Research Products-Ninth Ed., 2002 Molecular Probes, Eugene, Oreg.; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein).

Determination of the three-dimensional structures of representative antiviral polypeptides may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of antiviral polypeptides as provided herein, include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/.

Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

As generally referred to in the art, and as used herein, sequence identity and sequence homology may be used interchangeably and generally refer to the percentage of amino acid residues (or nucleotides) in a candidate sequence that are identical with, respectively, the amino acid residues (or nucleotides) in a reference polypeptide or polynucleotide sequence, after aligning the sequences and introducing gaps, if necessary, to achieve the maximum percent sequence identity, and optionally not considering any conservative substitutions as part of the sequence identity. In certain embodiments, a peptide such as an antiviral polypeptide of the embodiments disclosed herein shares at least about 75%, at least about 80%, at least about 85%, at least about 90%, 91%, 92%, 93% or 94%, or at least about 95%, 96%, 97%, 98%, or 99% of the amino acid residues (or of the nucleotides in a polynucleotide encoding such a peptide) with the sequence of the peptide of any one of SEQ ID NOS:1-155 or a peptide having the sequence of a general formula according to any one of formulae I-IV.

Such sequence identity may be determined according to well known sequence analysis algorithms, including those available from the University of Wisconsin Genetics Computer Group (Madison, Wis.), such as FASTA, Gap, Bestfit, BLAST, or others. In certain embodiments, the choice of amino acids in a functional antiviral peptide will depend in part on the physical, chemical and biological characteristics required of the peptide. The BLOSUM50 substitution matrix (Henikoff et al., 1992 Proc. Nat. Acad. Sci. USA 89:10915) and the natural biochemical properties of amino acids were used to identify conservative residues such that any of the following listed amino acids could be substituted for an equivalent residue in the peptides described in formula I-IV herein [SEQ ID NOS: 1-159] without substantially altering the peptide function: A=G, V, L, M, I, S; R=K, H, N, E, D, Q; N=D, H, S, K, R, E; D=N, E, K, R; C=S, T, Q; Q=R, E, H, K, S, T, C; E=D, Q, K, H, R, N; G=A, V, L, M, I; H=N, Q, Y, K, R, N, E, D; I=L, M, V, G, A; L=I, M, F, V, G, A; K=R, E, Q, H, N, D; M=I, L, V, G, A, M; F=L, W, Y; S=A, N, T, C, Q; T=S, C, Q; W=F, Y; Y=H, F, W; V=I, L, M, G, A, L. Amino acid equivalencies are set forth in Table 1.

TABLE 1
Amino acid equivalencies for selected antiviral peptides

| | Permissible Conservative |
|---|---|
| Amino Acid | Substitutions |
| A | G, A, V, L, M, I, or S |
| R | K, H, N, E, D, or Q |
| N | D, H, S, K, R, or E |
| D | N, E, K, or R |
| C | S, T, or Q |
| Q | R, E, H, K, S, T, or C |
| E | D, Q, K, H, R, or N |
| G | A, V, L, M, or I |
| H | N, Q, Y, K, R, N, E, or D |
| I | L, M, V, G, or A |
| L | I, M, F, V, G, or A |
| K | R, E, Q, H, N, or D |
| M | I, L, V, G, A, or M |
| F | L, W, or Y |
| P | P |
| S | A, N, T, C, or Q |
| T | S, C, or Q |
| W | F or Y |
| Y | H, F, or W |
| V | I, L, M, G, A, or L |

In certain other embodiments, permissible substitutions within the amino acid sequence of a herein disclosed antiviral polypeptide such as the polypeptides having the amino acid sequences set forth in any one of SEQ ID NOS: 1-155 are those provided by Yampolsky et al., 2005 Genet. 170:1459, herein incorporated by reference.

Representative examples of antiviral polypeptides according to the present disclosure are set forth in Table 2.

TABLE 2
Amino acid sequences of peptides with antiviral
activity

| | | Pep- |
|---|---|---|
| | | tide |
| SEQ ID NO | Peptide Sequence | Name |
| SEQ ID NO 1 | KQYSVTDALEDVNTSPQSTEEVVQSFLISQ | |
| SEQ ID NO 2 | HQYSVTDALEDVNTSPQSTEEVVQSFLISQ | |
| SEQ ID NO 3 | NQYSVTDALEDVNTSPQSTEEVVQSFLISQ | |
| SEQ ID NO 4 | RSYSVTDALEDVNTSPQSTEEVVQSFLISQ | |
| SEQ ID NO 5 | RTYSVTDALEDVNTSPQSTEEVVQSFLISQ | |
| SEQ ID NO 6 | RQFSVTDALEDVNTSPQSTEEVVQSFLISQ | |
| SEQ ID NO 7 | RQWSVTDALEDVNTSPQSTEEVVQSFLISQ | |
| SEQ ID NO 8 | RQYTVTDALEDVNTSPQSTEEVVQSFLISQ | |
| SEQ ID NO 9 | RQYQVTDALEDVNTSPQSTEEVVQSFLISQ | |
| SEQ ID NO 10 | RQYSATDALEDVNTSPQSTEEVVQSFLISQ | |
| SEQ ID NO 11 | RQYSGTDALEDVNTSPQSTEEVVQSFLISQ | |
| SEQ ID NO 12 | RQYSVSDALEDVNTSPQSTEEVVQSFLISQ | |
| SEQ ID NO 13 | RQYSVQDALEDVNTSPQSTEEVVQSFLISQ | |
| SEQ ID NO 14 | RQYSVTEALEDVNTSPQSTEEVVQSFLISQ | |
| SEQ ID NO 15 | RQYSVTDGLEDVNTSPQSTEEVVQSFLISQ | |
| SEQ ID NO 16 | RQYSVTDVLEDVNTSPQSTEEVVQSFLISQ | |
| SEQ ID NO 17 | RQYSVTDAIEDVNTSPQSTEEVVQSFLISQ | |
| SEQ ID NO 18 | RQYSVTDALDDVNTSPQSTEEVVQSFLISQ | |
| SEQ ID NO 19 | RQYSVTDALEEVNTSPQSTEEVVQSFLISQ | |
| SEQ ID NO 20 | RQYSVTDALEDANTSPQSTEEVVQSFLISQ | |
| SEQ ID NO 21 | RQYSVTDALEDGNTSPQSTEEVVQSFLISQ | |
| SEQ ID NO 22 | RQYSVTDALEDVKTSPQSTEEVVQSFLISQ | |
| SEQ ID NO 23 | RQYSVTDALEDVHTSPQSTEEVVQSFLISQ | |
| SEQ ID NO 24 | RQYSVTDALEDVRTSPQSTEEVVQSFLISQ | |
| SEQ ID NO 25 | RQYSVTDALEDVNSSPQSTEEVVQSFLISQ | |
| SEQ ID NO 26 | RQYSVTDALEDVNQSPQSTEEVVQSFLISQ | |
| SEQ ID NO 27 | RQYSVTDALEDVNTTPQSTEEVVQSFLISQ | |
| SEQ ID NO 28 | RQYSVTDALEDVNTQPQSTEEVVQSFLISQ | |
| SEQ ID NO 29 | RQYSVTDALEDVNTSPSSTEEVVQSFLISQ | |
| SEQ ID NO 30 | RQYSVTDALEDVNTSPTSTEEVVQSFLISQ | |
| SEQ ID NO 31 | RQYSVTDALEDVNTSPQTTEEVVQSFLISQ | |
| SEQ ID NO 32 | RQYSVTDALEDVNTSPQQTEEVVQSFLISQ | |
| SEQ ID NO 33 | RQYSVTDALEDVNTSPQSSEEVVQSFLISQ | |
| SEQ ID NO 34 | RQYSVTDALEDVNTSPQSQEEVVQSFLISQ | |
| SEQ ID NO 35 | RQYSVTDALEDVNTSPQSTDEVVQSFLISQ | |
| SEQ ID NO 36 | RQYSVTDALEDVNTSPQSTEDVVQSFLISQ | |
| SEQ ID NO 37 | RQYSVTDALEDVNTSPQSTEEAVQSFLISQ | |
| SEQ ID NO 38 | RQYSVTDALEDVNTSPQSTEEGVQSFLISQ | |
| SEQ ID NO 39 | RQYSVTDALEDVNTSPQSTEEVAQSFLISQ | |
| SEQ ID NO 40 | RQYSVTDALEDVNTSPQSTEEVGQSFLISQ | |
| SEQ ID NO 41 | RQYSVTDALEDVNTSPQSTEEVVSSFLISQ | |
| SEQ ID NO 42 | RQYSVTDALEDVNTSPQSTEEVVTSFLISQ | |
| SEQ ID NO 43 | RQYSVTDALEDVNTSPQSTEEVVQTFLISQ | |
| SEQ ID NO 44 | RQYSVTDALEDVNTSPQSTEEVVQQFLISQ | |
| SEQ ID NO 45 | RQYSVTDALEDVNTSPQSTEEVVQSWLISQ | |
| SEQ ID NO 46 | RQYSVTDALEDVNTSPQSTEEVVQSYLISQ | |
| SEQ ID NO 47 | RQYSVTDALEDVNTSPQSTEEVVQSFIISQ | |
| SEQ ID NO 48 | RQYSVTDALEDVNTSPQSTEEVVQSFLLSQ | |
| SEQ ID NO 49 | RQYSVTDALEDVNTSPQSTEEVVQSFLITQ | |
| SEQ ID NO 50 | RQYSVTDALEDVNTSPQSTEEVVQSFLIQQ | |
| SEQ ID NO 51 | RQYSVTDALEDVNTSPQSTEEVVQSFLISS | |
| SEQ ID NO 52 | RQYSVTDALEDVNTSPQSTEEVVQSFLIST | |
| SEQ ID NO 53 | RQYSVTDALEDVNTSPQSTEEVVQSFLISQ | CLS1N |
| SEQ ID NO 54 | RQYSVTDALEDVSTSPQSTEEVVQSFLISQ | CLS1S |
| SEQ ID NO 55 | GDVDVSAVQAKLGALELNQRDAAAETELRV | |
| SEQ ID NO 56 | VDVDVSAVQAKLGALELNQRDAAAETELRV | |
| SEQ ID NO 57 | AEVDVSAVQAKLGALELNQRDAAAETELRV | |
| SEQ ID NO 58 | ADADVSAVQAKLGALELNQRDAAAETELRV | |
| SEQ ID NO 59 | ADGDVSAVQAKLGALELNQRDAAAETELRV | |
| SEQ ID NO 60 | ADVEVSAVQAKLGALELNQRDAAAETELRV | |
| SEQ ID NO 61 | ADVDASAVQAKLGALELNQRDAAAETELRV | |
| SEQ ID NO 62 | ADVDGSAVQAKLGALELNQRDAAAETELRV | |
| SEQ ID NO 63 | ADVDVTAVQAKLGALELNQRDAAAETELRV | |
| SEQ ID NO 64 | ADVDVQAVQAKLGALELNQRDAAAETELRV | |
| SEQ ID NO 65 | ADVDVSGVQAKLGALELNQRDAAAETELRV | |
| SEQ ID NO 66 | ADVDVSVVQAKLGALELNQRDAAAETELRV | |
| SEQ ID NO 67 | ADVDVSAAQAKLGALELNQRDAAAETELRV | |
| SEQ ID NO 68 | ADVDVSAGQAKLGALELNQRDAAAETELRV | |
| SEQ ID NO 69 | ADVDVSAVSAKLGALELNQRDAAAETELRV | |
| SEQ ID NO 70 | ADVDVSAVTAKLGALELNQRDAAAETELRV | |
| SEQ ID NO 71 | ADVDVSAVQGKLGALELNQRDAAAETELRV | |
| SEQ ID NO 72 | ADVDVSAVQVKLGALELNQRDAAAETELRV | |
| SEQ ID NO 73 | ADVDVSAVQAHLGALELNQRDAAAETELRV | |
| SEQ ID NO 74 | ADVDVSAVQARLGALELNQRDAAAETELRV | |
| SEQ ID NO 75 | ADVDVSAVQANLGALELNQRDAAAETELRV | |
| SEQ ID NO 76 | ADVDVSAVQAKIGALELNQRDAAAETELRV | |
| SEQ ID NO 77 | ADVDVSAVQAKLAALELNQRDAAAETELRV | |
| SEQ ID NO 78 | ADVDVSAVQAKLVALELNQRDAAAETELRV | |
| SEQ ID NO 79 | ADVDVSAVQAKLGGLELNQRDAAAETELRV | |
| SEQ ID NO 80 | ADVDVSAVQAKLGVLELNQRDAAAETELRV | |
| SEQ ID NO 81 | ADVDVSAVQAKLGAIELNQRDAAAETELRV | |
| SEQ ID NO 82 | ADVDVSAVQAKLGALDLNQRDAAAETELRV | |
| SEQ ID NO 83 | ADVDVSAVQAKLGALEINQRDAAAETELRV | |
| SEQ ID NO 84 | ADVDVSAVQAKLGALELKQRDAAAETELRV | |
| SEQ ID NO 85 | ADVDVSAVQAKLGALELHQRDAAAETELRV | |
| SEQ ID NO 86 | ADVDVSAVQAKLGALELRQRDAAAETELRV | |
| SEQ ID NO 87 | ADVDVSAVQAKLGALELNSRDAAAETELRV | |
| SEQ ID NO 88 | ADVDVSAVQAKLGALELNTRDAAAETELRV | |
| SEQ ID NO 89 | ADVDVSAVQAKLGALELNQKDAAAETELRV | |
| SEQ ID NO 90 | ADVDVSAVQAKLGALELNQHDAAAETELRV | |
| SEQ ID NO 91 | ADVDVSAVQAKLGALELNQNDAAAETELRV | |
| SEQ ID NO 92 | ADVDVSAVQAKLGALELNQREAAAETELRV | |
| SEQ ID NO 93 | ADVDVSAVQAKLGALELNQRDGAAETELRV | |
| SEQ ID NO 94 | ADVDVSAVQAKLGALELNQRDVAAETELRV | |
| SEQ ID NO 95 | ADVDVSAVQAKLGALELNQRDAGAETELRV | |
| SEQ ID NO 96 | ADVDVSAVQAKLGALELNQRDAVAETELRV | |
| SEQ ID NO 97 | ADVDVSAVQAKLGALELNQRDAAGETELRV | |
| SEQ ID NO 98 | ADVDVSAVQAKLGALELNQRDAAVETELRV | |
| SEQ ID NO 99 | ADVDVSAVQAKLGALELNQRDAAADTELRV | |
| SEQ ID NO 100 | ADVDVSAVQAKLGALELNQRDAAAESELRV | |
| SEQ ID NO 101 | ADVDVSAVQAKLGALELNQRDAAAEQELRV | |
| SEQ ID NO 102 | ADVDVSAVQAKLGALELNQRDAAAETDLRV | |
| SEQ ID NO 103 | ADVDVSAVQAKLGALELNQRDAAAETEIRV | |
| SEQ ID NO 104 | ADVDVSAVQAKLGALELNQRDAAAETELKV | |
| SEQ ID NO 105 | ADVDVSAVQAKLGALELNQRDAAAETELHV | |
| SEQ ID NO 106 | ADVDVSAVQAKLGALELNQRDAAAETELNV | |
| SEQ ID NO 107 | ADVDVSAVQAKLGALELNQRDAAAETELRA | |
| SEQ ID NO 108 | ADVDVSAVQAKLGALELNQRDAAAETELRG | |
| SEQ ID NO 109 | ADVDVSAVQAKLGALELNQRDAAAETELRV | CLS2A |
| SEQ ID NO 110 | ADTVGLIDEQNEASKTNGLGAAEAF | |
| SEQ ID NO 111 | VDTVGLIDEQNEASKTNGLGAAEAF | |
| SEQ ID NO 112 | GETVGLIDEQNEASKTNGLGAAEAF | |
| SEQ ID NO 113 | GDSVGLIDEQNEASKTNGLGAAEAF | |
| SEQ ID NO 114 | GDQVGLIDEQNEASKTNGLGAAEAF | |
| SEQ ID NO 115 | GDTAGLIDEQNEASKTNGLGAAEAF | |
| SEQ ID NO 116 | GDTGGLIDEQNEASKTNGLGAAEAF | |
| SEQ ID NO 117 | GDTVALIDEQNEASKTNGLGAAEAF | |
| SEQ ID NO 118 | GDTVVLIDEQNEASKTNGLGAAEAF | |
| SEQ ID NO 119 | GDTVGIIDEQNEASKTNGLGAAEAF | |
| SEQ ID NO 120 | GDTVGLLDEQNEASKTNGLGAAEAF | |
| SEQ ID NO 121 | GDTVGLIEEQNEASKTNGLGAAEAF | |
| SEQ ID NO 122 | GDTVGLIDDQNEASKTNGLGAAEAF | |
| SEQ ID NO 123 | GDTVGLIDESNEASKTNGLGAAEAF | |
| SEQ ID NO 124 | GDTVGLIDETNEASKTNGLGAAEAF | |
| SEQ ID NO 125 | GDTVGLIDEQKEASKTNGLGAAEAF | |
| SEQ ID NO 126 | GDTVGLIDEQHEASKTNGLGAAEAF | |
| SEQ ID NO 127 | GDTVGLIDEQREASKTNGLGAAEAF | |
| SEQ ID NO 128 | GDTVGLIDEQNDASKTNGLGAAEAF | |
| SEQ ID NO 129 | GDTVGLIDEQNEGSKTNGLGAAEAF | |
| SEQ ID NO 130 | GDTVGLIDEQNEVSKTNGLGAAEAF | |
| SEQ ID NO 131 | GDTVGLIDEQNEATKTNGLGAAEAF | |
| SEQ ID NO 132 | GDTVGLIDEQNEAQKTNGLGAAEAF | |
| SEQ ID NO 133 | GDTVGLIDEQNEASHTNGLGAAEAF | |
| SEQ ID NO 134 | GDTVGLIDEQNEASRTNGLGAAEAF | |
| SEQ ID NO 135 | GDTVGLIDEQNEASNTNGLGAAEAF | |
| SEQ ID NO 136 | GDTVGLIDEQNEASKSNGLGAAEAF | |
| SEQ ID NO 137 | GDTVGLIDEQNEASKQNGLGAAEAF | |
| SEQ ID NO 138 | GDTVGLIDEQNEASKTKGLGAAEAF | |
| SEQ ID NO 139 | GDTVGLIDEQNEASKTHGLGAAEAF | |
| SEQ ID NO 140 | GDTVGLIDEQNEASKTRGLGAAEAF | |
| SEQ ID NO 141 | GDTVGLIDEQNEASKTNALGAAEAF | |
| SEQ ID NO 142 | GDTVGLIDEQNEASKTNVLGAAEAF | |
| SEQ ID NO 143 | GDTVGLIDEQNEASKTNGIGAAEAF | |
| SEQ ID NO 144 | GDTVGLIDEQNEASKTNGLAAAEAF | |
| SEQ ID NO 145 | GDTVGLIDEQNEASKTNGLVAAEAF | |
| SEQ ID NO 146 | GDTVGLIDEQNEASKTNGLGGAEAF | |
| SEQ ID NO 147 | GDTVGLIDEQNEASKTNGLGVAEAF | |
| SEQ ID NO 148 | GDTVGLIDEQNEASKTNGLGAGEAF | |
| SEQ ID NO 149 | GDTVGLIDEQNEASKTNGLGAVEAF | |
| SEQ ID NO 150 | GDTVGLIDEQNEASKTNGLGAADAF | |
| SEQ ID NO 151 | GDTVGLIDEQNEASKTNGLGAAEGF | |
| SEQ ID NO 152 | GDTVGLIDEQNEASKTNGLGAAEVF | |
| SEQ ID NO 153 | GDTVGLIDEQNEASKTNGLGAAEAW | |
| SEQ ID NO 154 | GDTVGLIDEQNEASKTNGLGAAEAY | |
| SEQ ID NO 155 | GDTVGLIDEQNEASKTNGLGAAEAF | CLS2G |

As generally referred to in the art, and as used herein, the term subject refers to humans or other mammals or vertebrates, or to plants including agricultural species, in which a targeted virus exhibits tropism and said virus is inhibited by at least one antiviral activity of the peptides as set forth in any one of SEQ ID NOS: 1-159. It is within the art for a person skilled in the relevant medical, veterinary, botanical and/or agricultural sciences to determine that a human, animal or plant subject has a viral infection or may be suspected of, or at risk for, having a viral infection, using methodologies that are described in standard texts such as Foreign Animal Diseases, seventh edition, Boca Publishing Group, Boca Raton, Fla., ISBN 978-0-9659583-4-9 (2008), or Lennette's Laboratory Diagnosis of Viral Infections, fourth edition, Informa Healthcare, Inc, USA ISBN-13: 978-1420084955 (2010), or Plant Pathology: Techniques and Protocols, Robert Burns, ed., Human Press, NY ISBN-13: 978-1588297990 (2009), and other like references.

The presently disclosed antiviral polypeptides may, according to certain embodiments, usefully be contacted with a cell in vivo or in vitro or with a subject, the cell or the subject having, or being at risk for having or suspected of having, a viral infection, according to a herein disclosed method of substantially impairing a viral activity in a cell. Such presence or risk of viral infection may be determined according to criteria that are well known in the art (e.g., as noted above). The viral activity that is substantially impaired may comprise at least one of (i) binding of a virus to a cell to which the virus exhibits tropism; (ii) fusion of a virus to a cell membrane of a cell to which the virus exhibits tropism; (iii) viral entry by a virus into a cell to which the virus exhibits tropism; (iv) viral replication or viral assembly by a virus in a cell to which the virus exhibits tropism; (v) release from a virus-infected cell of viral particles that have been synthesized in the cell as a result of infection by the virus; and (vi) lysis of a virus-infected cell that results from infection of the cell by the virus. Persons familiar with the art will be familiar with methodologies and criteria with which it can be determined when a viral activity has been substantially impaired, as described herein.

Certain embodiments are thus directed to a pharmaceutical composition comprising any one or more of the herein disclosed antiviral polypeptides (e.g., a polypeptide having the amino acid sequence set forth in SEQ ID NOS:1-159, or a chemically modified or allelic variant thereof); and a pharmaceutical carrier or excipient. The pharmaceutical compositions can be prepared by combining an antiviral polypeptide or antiviral polypeptide-containing composition with an appropriate physiologically acceptable carrier, diluent or excipient, and may be formulated into preparations in solid, semi-solid, liquid or microparticle—(e.g., microdroplet) containing gaseous forms, such as tablets, capsules, powders, granules, ointments, solutions, suppositories, injections, inhalants, gels, microspheres, and aerosols. In addition, other pharmaceutically active ingredients and/or suitable excipients such as salts, buffers and stabilizers may, but need not, be present within the composition. Administration may be achieved by a variety of different routes, including oral, parenteral, nasal, intravenous, intradermal, subcutaneous or topical.

Preferred modes of administration depend upon the nature of the condition to be treated or prevented, which in certain embodiments will refer to a deleterious or clinically undesirable condition the extent, severity, likelihood of occurrence and/or duration of which may be decreased (e.g., reduced in a statistically significant manner relative to an appropriate control situation such as an untreated control) according to certain methods provided herein. An amount that, following administration, detectably reduces, inhibits, prevents, decreases the severity or likelihood of occurrence of, or delays such a condition, for instance, the onset or exacerbation of a viral infection, disease or disorder in a human, an animal, or in a plant is considered a therapeutically effective amount. Persons skilled in the relevant arts will be familiar with any number of diagnostic, surgical and/or other clinical, veterinary, botanical and/or agricultural criteria that may indicate the appropriateness of, and/or to which can be adapted, administration of the antiviral polypeptide and peptide compositions described herein.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal, intrathecal, injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described antiviral polypeptide in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an antiviral polypeptide of the present disclosure, for treatment of a disease or condition of interest in accordance with teachings herein.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of an antiviral polypeptide as herein disclosed such that a suitable dosage will be obtained. Typically, this amount is at least 0.01% of the antiviral polypeptide in the composition. When intended for oral administration, this amount may be varied to be between 0.1 and about 70% of the weight of the composition. Certain oral pharmaceutical compositions contain between about 4% and about 75% of the antiviral polypeptide. In certain embodiments, pharmaceutical compositions and preparations according to the present invention are prepared so that a parenteral dosage unit contains between 0.01 to 10% by weight of the antiviral polypeptide prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

Certain preferred embodiments contemplate such topical formulations of the herein described antiviral polypeptides, including embodiments in which high local concentrations of the antiviral peptides may be desired in order to obtain a therapeutically effective amount. Certain related embodiments, for example, may involve topical administration and/or administration in or around urogenital or anal areas as may be useful for treating or reducing the likelihood of occurrence or severity of sexually transmitted viral diseases, including human immunodeficiency virus (HIV) and/or herpes virus infections. Animal models are known for testing safety and efficacy of topical antiviral formulations, as described, for example, by Shipman, C., J R, Smith, S. H., Drach, J. C. and Klayman, D. L. (1986) Thiosemicarbazones of 2-acetylpyridine, 2-acetylquinoline, 1-acetylisoquinoline and related compounds as inhibitors of herpes simplex virus in vitro and in a cutaneous herpes guinea pig model. Antiviral Research 6:197-222.

The pharmaceutical composition may in certain embodiments include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the antiviral polypeptide and thereby assists in the delivery of the compound. Suitable agents that may act in this capacity include monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises an antiviral polypeptide as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antiviral polypeptide in the aqueous delivery system.

The compositions are administered in a therapeutically effective amount, which will vary depending upon a variety of factors including the activity of the specific antiviral polypeptide compound that is employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

It will be appreciated that the practice of the several embodiments of the present invention will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y.(2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

### EXAMPLES

### Example 1

### Anti HSV1 Activity of the CLS1N Peptide

The exemplary peptide CLS1N having the amino acid sequence set forth as SEQ ID NO:53, was synthesized using solid phase synthesis (Stewart and Young, 1969) and a standard procedure of Fmoc-(9-fluorenylmethyloxycarbonyl) N-terminal alpha-amino protection and PyBOP (Benzotriazole-1-yl-oxy-tris-pyrrolidino-phosphonium hexafluorophosphate) as the activation reagent on a Symphony peptide synthesizer (Protein Technologies, Inc.) with TentaGel R or S RAM resin of Rapp Polymer. After cleavage of the peptide from the resin with Trifluoroacetic acid (TFA), 3% 1,2-Ethanedithiol and 4% Triisopropylsilane, the peptide was purified by preparative HPLC. The change of counter-ion from TFA to chloride was performed using acetonitrile with 0.05% HCl during the HPLC purification. Peptide identity and purity were analyzed by mass spectrometry coupled with analytical HPLC using the LC/MSD Trap series 1100 system (Agilent Technologies) in combination with a Phenomenex Gemini-NX column. The peptides were then further modified from the native form by acetylation of the carboxy terminus and by amidation of the amino terminus.

Feasibly, peptides could also be synthesized by another method well known in art for example, by partial solid-phase techniques, or by fragment condensation, or by classical solution couplings, or by recombinant genetics followed by protein expression and purification. A common feature of the synthesis chemistries is the protection of the side-chain groups during the sequential extension of the oligopeptide by the addition of modified amino acid residues with suitable protecting groups that prevent the chemical reaction from aberrantly occurring at that site until the group is removed. Consequently, during synthesis an intermediate nascent oligopeptide is formed with the desired amino acid residues in the appropriate sequence with the side-chain protecting groups still attached. Since the chemical synthesis of polypeptides is less than 100% efficient a measurable amount of synthesized peptide will retain the protective side groups, and will not be fully extended and could be co-purified with the full length peptide. Therefore these intermediate compounds are included within the scope of certain contemplated embodiments.

One method to demonstrate inhibition of viral replication is the plaque assay which is well known in the art and described in texts such as Kaufmann, S. H.; Kabelitz, D. (2002) Methods in Microbiology Vol.32: Immunology of Infection. Academic Press. ISBN 0-12-521532-0. In this example, the CLS1N peptide effectively inhibited virus growth in cells using the plaque inhibition assay. Peptide CLS1N was tested for the ability to inhibit replication of herpes simplex virus type 1 strain KOS in Vero African green monkey kidney cells, with acyclovir as a control. Peptide was re-solubilized in 500 μl of 200 mM sodium phosphate buffer, pH 7.2 with 10% molecular grade DMSO, then diluted to 600 μg/ml with 0.4% DMSO and 20 mM phosphate to prepare three additional half-log serial dilutions, in three replicates per concentration, in DMEM tissue culture medium, 2% fetal bovine serum and 0.4% DMSO, penicillin/streptomycin antibiotic.

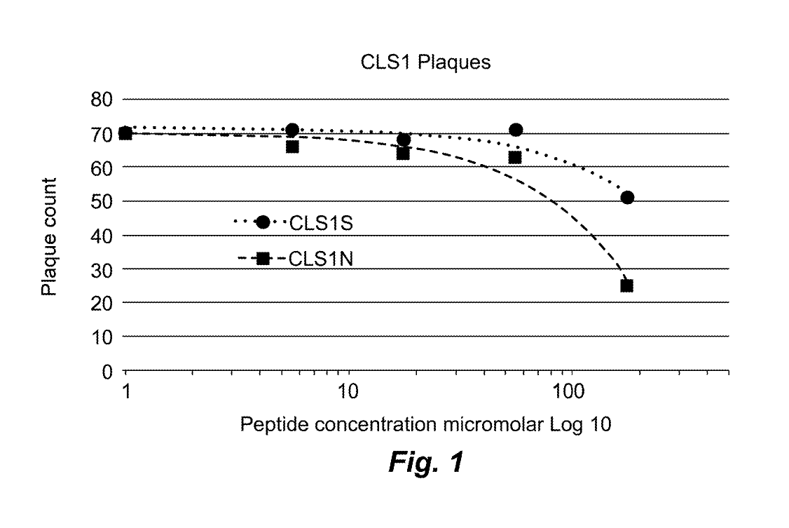

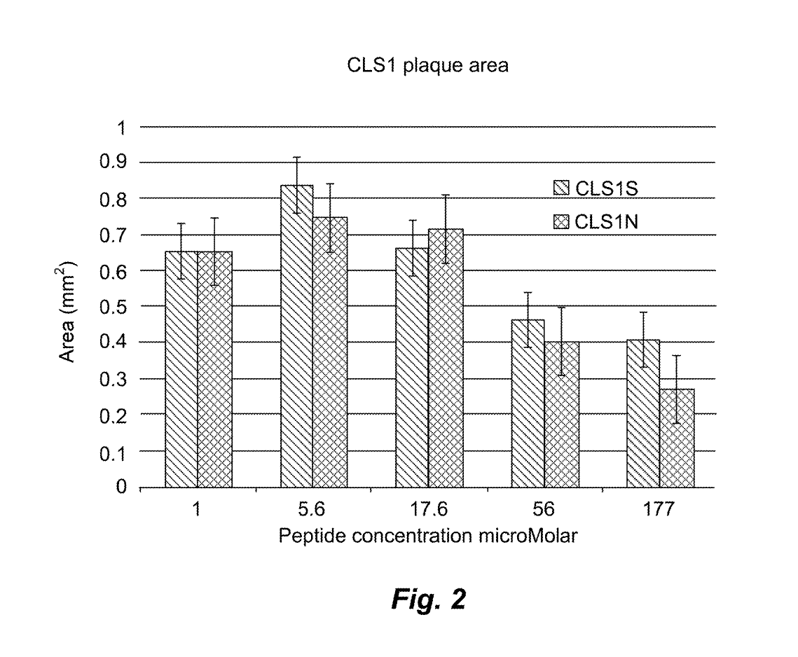

Vero cells were plated at 1×10⁵ per cm² in DMEM media in 6-well plates 18 hours prior to adding drug dilutions. Growth media was removed from each prepared cell well and 100 μl of each drug dilution was added. After incubating for one hour at 37° C., approximately 60 plaque forming units of herpes simplex virus type 1 strain KOS were added per well. Virus was permitted to adsorb to the cells for two hours and then the media were aspirated from the monolayers and replaced with media containing dilutions of peptide. After incubating three days at 37° C. Vero monolayers were fixed and stained with crystal violet and photographed for plaque evaluation. See FIGS. 1 and 2.

The CLS1N peptide [SEQ ID NO:53] at a concentration of 177 μM caused moderate thinning and moderately less intense staining of the monolayer as compared to cell control wells. Plaques were still visible on this monolayer however, although reduced in number. Dilution to 56 μM did not affect the density of the monolayer and was graded as no cytotoxicity. The plaque size in this treatment was pinpoint (approx. 0.7 mm diameter). These plaques were approximately the same size as those in the monolayer treated with 5 μM acyclovir. Untreated plaques were approximately 1.5 mm diameter. The plaques in dilutions 3 and 4 at 17.6 μM and 5.6 μM, respectively, were of wild type size. There was not significant inhibition of plaque number as compared to media-only treated controls. The monolayers in the plate containing dilutions 3 and 4 were normal microscopically.

### Example 2

### Anti HSV1 Activity of CLS1S Peptide

Another exemplary polypeptide, termed CLS1S peptide and having the amino acid sequence set forth as SEQ ID NO:54, was synthesized using solid phase synthesis on a Symphony peptide synthesizer as described above, then purified to 80% purity using HPLC. Peptides were diluted serially in half-log dilutions then added to Vero cells pre-incubated with HSV1 KOS at a multiplicity of infection as described above for CLS1N [SEQ ID NO:53].

### Results

At the highest concentration the CLS1S peptide [SEQ ID NO:54] caused moderate thinning of the Vero cell monolayer as compared to control untreated cells. Plaques were still visible, although reduced in number compared to the untreated control cells. The 188 ug/ml dilution did not affect the density of the monolayer and plaques were reduced to approximately half the diameter of untreated plaques and were approximately the same size as in the monolayer treated with 50 uM acyclovir. Plaques at the lower concentrations were of wild-type size and there was no measurable reduction in plaque number. See FIGS. 1 and 2.

### Example 3

### CLS2A and CLS2G Inhibition of Influenza H3N2

Two additional exemplary antiviral polypeptides according to the present disclosure, the distinct CLS2A [SEQ ID NO:109] and CLS2G [SEQ ID NO:155] peptides corresponding to different regions of a protein encoded by a candidate antiviral survival gene, were prepared using solid phase synthesis followed by HPLC purification as described above. Desiccated peptides were solubilized in 200mM sodium phosphate, pH 7.2 with 2% tissue culture grade DMSO. Half-log serial dilutions were prepared in DMEM as described above. MDCK canine kidney cells were plated at 7×10⁴ cells per cm² in 6 well plates and following 18 hours incubation at 37 C, growth media were removed and 100 plaque forming units of influenza H3N2/Wisconsin/67/2005 were added per well. Virus was permitted to absorb to the cells for two hours following which the media were aspirated and replaced with influenza growth media containing the dilutions of the peptide in three replicates for each concentration. After 48 hours of incubation at 35° C., monolayers were stained with vital dye and photographed. Images were processed for plaque counts and plaque area using ImageJ software.

### Results

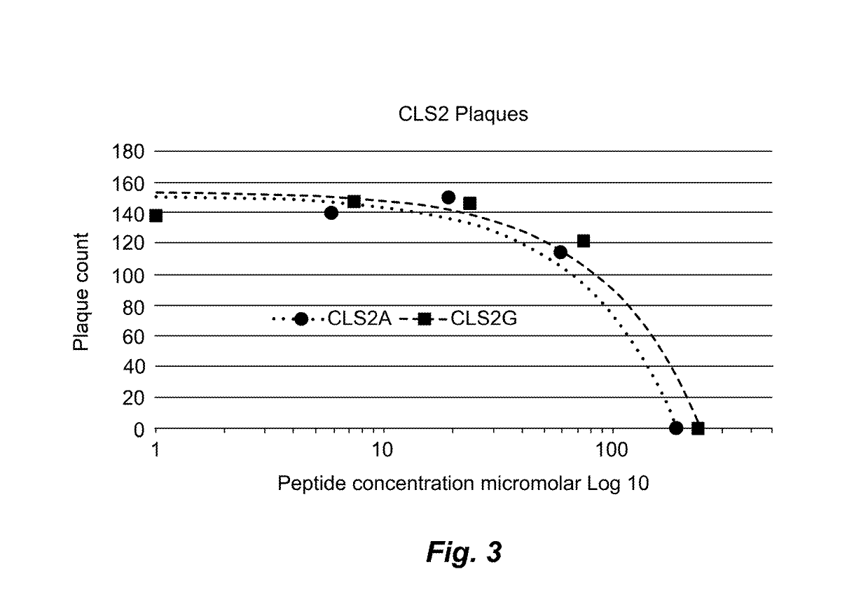

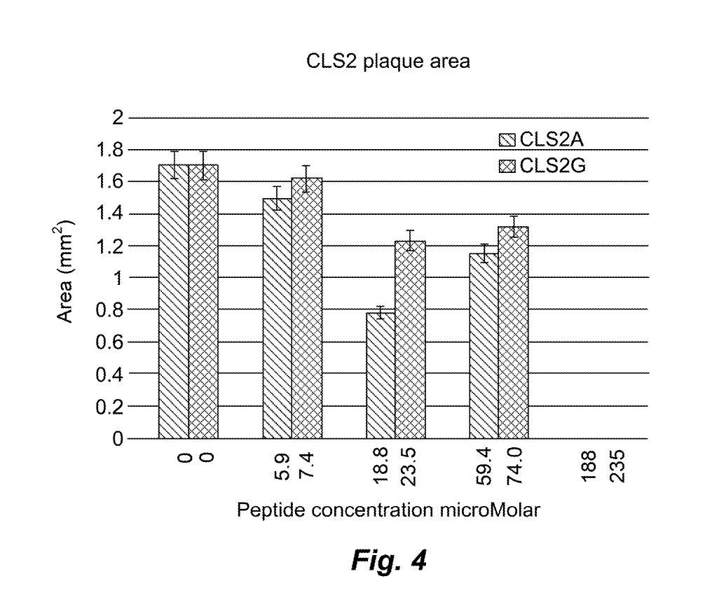

At the highest concentration tested (188 μM for CLS2A [SEQ ID NO:109] and 235 μM for CLS2G [SEQ ID NO:155]) both peptides caused moderate thinning and more intense staining of the monolayer as compared to cell control wells with no treatment. Plaques were not visible on this monolayer, an indication that the monolayer was not healthy as a result of drug treatment. At the second concentration (59.4 μM for CLS2A and 74.0 μM for CLS2G), both peptides exhibited no cytotoxicity. The plaque sizes in this treatment were reduced in area and this treatment also had a slight reduction in the number of H3N2 plaques formed. At the 18.8 μM concentration plaque areas were reduced in area compared to control untreated cells with the CLS2A peptide showing a greater effect compared to CLS2G. Plaque counts at this concentration were similar to the untreated control. At the 5.9 to 7.4 μM concentration both peptides produced plaques of similar size and number as in the untreated virus control. See FIGS. 3 and 4. The control drug Oseltamivir was used at 10 μM concentration and was completely inhibitory.

### SELECTED REFERENCES

U.S. Pat. No. 7,371,809 B2

U.S. Pat. No. 5,441,966

U.S. Pat. No. 4,795,740

U.S. Pat. No. 8,592,552 B2

US 2010/0041604 A1

Gilbert et al, Avian flu and climate change, Rev Sci Tech. 2008 August; 27(2): 459-46. 6

Sally A. Lahm, Maryvonne Kombila, Robert Swanepoel, Richard F. W. Barnes, Morbidity and mortality of wild animals in relation to outbreaks of Ebola haemorrhagic fever in Gabon, 1994-2003, Transactions of the Royal Society of Tropical Medicine and Hygiene (2007) 101, 64-78.

Peterson, A. T., Bauer, J. T., Mills, J. N., 2004. Ecologic and geographic distribution of filovirus disease. Emerg. Infect. Dis. 10, 40-47.

Pinzon, J. E., Wilson, J. M., Tucker, C. J., Arthur, R., Jahrling, P. B., Formenty, P., 2004. Trigger events: enviroclimatic coupling of Ebola haemorrhagic fever outbreaks. Am. J. Trop. Med. Hyg. 71, 664-674.

Tucker, C J, Wilson, J M M, Mahoney, R, Anyamba, A, Linthicum, K, Myers, M F, 2002. Climatic and ecological context of the 1994-1996 Ebola outbreaks. Photogr. Engin. Remote Sens. 2, 147-152.

Stone, R, Is Live Smallpox Lurking in the Arctic?, Science 15 March 2002: Vol. 295 no. 5562 pp. 2002DOI:10.1126/science.295.5562.2002.

Stewart, J M and Young, J D, ‘Solid-Phase Peptide Synthesis’, W. H. Freeman and Company, San Francisco, 1969, pp. 40-49.

Smith J S, Robinson N J (2002). “Age-specific prevalence of infection with herpes simplex virus types 2 and 1: a global review”. J. Infect. Dis. 186 Suppl 1: S3-28. doi:10.1086/343739. PMID 12353183.

Tsetsarkin K A, Vanlandingham D L, McGee C E, Higgs S (2007). “A Single Mutation in Chikungunya Virus Affects Vector Specificity and Epidemic Potential”. PLoS Pathog 3 (12): e201. doi:10.1371/journal.ppat.0030201. PMC 2134949. PMID 18069894.

Ebell, M H; Call, M; Shinholser, J (April 2013). “Effectiveness of oseltamivir in adults: a meta-analysis of published and unpublished clinical trials.”. Family practice 30 (2): 125-33. doi:10.1093/fampra/cms059. PMID 22997224.

Henikoff, S. and Henikoff, J. G. Amino acid substitution matrices from protein blocks Proc. Natl. Acad. Sci. USA 89, 10915-10919 (1992)

Centers for Disease Control and Prevention (CDC) (2006). “High levels of adamantane resistance among influenza A (H3N2) viruses and interim guidelines for use of antiviral agents—United States, 2005-06 influenza season”. MMWR Morb Mortal Wkly Rep 55 (2): 44-6. PMID 16424859.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antiviral polypeptide of at least 15, 16, 17, 18, 19, 20, 21, 22, 23, 24, or 25 amino acids and not more than 50, 49, 48, 47, 46, 45, 44, 43, 42, 41, 40, 39, 38, 37, 36, 35, 34, 33, 32, 31 or 30 amino acids, comprising a peptide of general formula I:
N-X-C [I] wherein:
(a) N is an amino terminus of the antiviral polypeptide and either
(1) N consists of 0, 1, 2, 3, 4, 5, 6, 7, 8, 9, 10, 11, 12, 13, 14, 15, 16, 17, 18, 19, or 20 amino acids that are independently selected from natural and non-natural amino acids, or
(2) N is an amino terminus of the antiviral polypeptide of general formula II:
N1-N2 [II] wherein:
N1 is a non-natural amino acid and N2 consists of 0, 1, 2, 3, 4, 5, 6, 7, 8, 9, 10, 11, 12, 13, 14, 15, 16, 17, 18, or 19 amino acids that are independently selected from natural and non-natural amino acids;
(b) C is a carboxy terminus of the antiviral polypeptide and either
(1) C consists of 0, 1, 2, 3, 4, 5, 6, 7, 8, 9, 10, 11, 12, 13, 14, 15, 16, 17, 18, 19, or 20 amino acids that are independently selected from natural and non-natural amino acids, or
(2) C is a carboxy terminus of the antiviral polypeptide of general formula II:
C1-C2 [II] wherein:
C1 consists of 0, 1, 2, 3, 4, 5, 6, 7, 8, 9, 10, 11, 12, 13, 14, 15, 16, 17, 18, or 19 amino acids that are independently selected from natural and non-natural amino acids and C2 is a non-natural amino acid; and
(c) X is a peptide of 30, 29, 28, 27, 26, 25, 24, 23, 22, 21, 20, 19, 18, 17, 16, or 15 amino acids and X is one of:
(1) a peptide of general formula III:
[III]
[SEQ ID NO: 156]
X1-X2-X3-X4-X5-X6-X7-X8-X9-X10-X11-X12-X13-X14-
X15-X16-X17-X18-X19-X20-X21-X22-X23-X24-X25-X26-
X27-X28-X29-X30
wherein:
X1 is R, K, H, N, E, D, or Q;
X2 is Q, R, E, H, K, S, T, or C;
X3 is Y, H, F, or W;
X4 is S, A, N, T, C, or Q;
X5 is V, I, L, M, G, A, or L;
X6 is T, S, C, or Q;
X7 is D, N, E, K, or R;
X8 is G, A, V, L, M, I, or S;
X9 is L, I, M, F, V, G, or A;
X10 is E, D, Q, K, H, R, or N;
X11 is D, N, E, K, or R;
X12 is Y, H, F, or W;
X13 is N, D, H, S, K, R, or E;
X14 is T, S, C, or Q;
X15 is S, A, N, T, C, or Q;
X16 is P;
X17 is Q, R, E, H, K, S, T, or C;
X18 is S, A, N, T, C, or Q;
X19 is T, S, C, or Q;
X20 is E, D, Q, K, H, R, or N;
X21 is E, D, Q, K, H, R, or N;
X22 is V, I, L, M, G, A, or L;
X23 is V, I, L, M, G, A, or L;
X24 is Q, R, E, H, K, S, T, or C;
X25 is S, A, N, T, C, or Q;
X26 is F, L, W, or Y;
X27 is L, I, M, F, V, G, or A;
X28 is I, L, M, V, G, or A;
X29 is S, A, N, T, C, or Q;
X30 is Q, R, E, H, K, S, T, or C;
(2) a peptide of general formula IV:
[IV]
[SEQ ID NO: 157]
X1-X2-X3-X4-X5-X6-X7-X8-X9-X10-X11-X12-X13-X14-
X15-X16-X17-X18-X19-X20-X21-X22-X23-X24-X25-X26-
X27-X28-X29-X30
wherein
X1 is R, K, H, N, E, D, or Q;
X2 is Q, R, E, H, K, S, T, or C;
X3 is Y, H, F, or W;
X4 is S, A, N, T, C, or Q;
X5 is V, I, L, M, G, A, or L;
X6 is T, S, C, or Q;
X7 is D, N, E, K, or R;
X8 is G, A, V, L, M, I, or S;
X9 is L, I, M, F, V, G, or A;
X10 is E, D, Q, K, H, R, or N;
X11 is D, N, E, K, or R;
X12 is Y, H, F, or W;
X13 is S, A, N, T, C, or Q;
X14 is T, S, C, or Q;
X15 is S, A, N, T, C, or Q;
X16 is P;
X17 is Q, R, E, H, K, S, T, or C;
X18 is S, A, N, T, C, or Q;
X19 is T, S, C, or Q;
X20 is E, D, Q, K, H, R, or N;
X21 is E, D, Q, K, H, R, or N;
X22 is V, I, L, M, G, A, or L;
X23 is V, I, L, M, G, A, or L;
X24 is Q, R, E, H, K, S, T, or C;
X25 is S, A, N, T, C, or Q;
X26 is F, L, W, or Y;
X27 is L, I, M, F, V, G, or A;
X28 is I, L, M, V, G, or A;
X29 is S, A, N, T, C, or Q;
X30 is Q, R, E, H, K, S, T, or C;
(3) a peptide of general formula V:
[V]
[SEQ ID NO: 158]
X1-X2-X3-X4-X5-X6-X7-X8-X9-X10-X11-X12-X13-X14-X15-
X16-X17-X18-X19-X20-X21-X22-X23-X24-X25-X26-X27-
X28-X29-X30
wherein
X1 is A, G, A, V, L, M, I, or S;
X2 is D, N, E, K, or R;
X3 is V, I, L, M, G, A, or L;
X4 is D, N, E, K, or R;
X5 is V, I, L, M, G, A, or L;
X6 is S, A, N, T, C, or Q;
X7 is A, G, A, V, L, M, I, or S;
X8 is V, I, L, M, G, A, or L;
X9 is Q, R, E, H, K, S, T, or C;
X10 is A, G, A, V, L, M, I, or S;
X11 is K, R, E, Q, H, N, or D;
X12 is L, I, M, F, V, G, or A;
X13 is G, A, V, L, M, or I;
X14 is A, G, A, V, L, M, I, or S;
X15 is L, I, M, F, V, G, or A;
X16 is E, D, Q, K, H, R, or N;
X17 is L, I, M, F, V, G, or A;
X18 is N, D, H, S, K, R, or E;
X19 is Q, R, E, H, K, S, T, or C;
X20 is R, K, H, N, E, D, or Q;
X21 is D, N, E, K, or R;
X22 is A, G, A, V, L, M, I, or S;
X23 is A, G, A, V, L, M, I, or S;
X24 is A, G, A, V, L, M, I, or S;
X25 is E, D, Q, K, H, R, or N;
X26 is T, S, C, or Q;
X27 is E, D, Q, K, H, R, or N;
X28 is L, I, M, F, V, G, or A;
X29 is R, K, H, N, E, D, or Q;
X30 is V, I, L, M, G, A, or L;
(4) a peptide of general formula VI:
[VI]
[SEQ ID NO: 159]
X1-X2-X3-X4-X5-X6-X7-X8-X9-X10-X11-X12-X13-X14-X15-
X16-X17-X18-X19-X20-X21-X22-X23-X24-X25
wherein:
X1 is G, A, V, L, M, or I;
X2 is D, N, E, K, or R;
X3 is T, S, C, or Q;
X4 is V, I, L, M, G, A, or L;
X5 is G, A, V, L, M, or I;
X6 is L, I, M, F, V, G, or A;
X7 is I, L, M, V, G, or A;
X8 is D, N, E, K, or R;
X9 is E, D, Q, K, H, R, or N;
X10 is Q, R, E, H, K, S, T, or C;
X11 is N, D, H, S, K, R, or E;
X12 is E, D, Q, K, H, R, or N;
X13 is A, G, A, V, L, M, I, or S;
X14 is S, A, N, T, C, or Q;
X15 is K, R, E, Q, H, N, or D;
X16 is T, S, C, or Q;
X17 is N, D, H, S, K, R, or E;
X18 is G, A, V, L, M, or I;
X19 is L, I, M, F, V, G, or A;
X20 is G, A, V, L, M, or I;
X21 is A, G, A, V, L, M, I, or S;
X22 is A, G, A, V, L, M, I, or S;
X23 is E, D, Q, K, H, R, or N;
X24 is A, G, A, V, L, M, I, or S;
X25 is F, L, W, or Y;
(5) a peptide that comprises the amino acid sequence set forth in any one of SEQ ID NOS:1-155,
(6) a peptide that comprises 15, 16, 17, 18, 19, 20, 21, 22, 23, 24, 25, 26, 27, 28, 29, or 30 contiguous amino acids of the amino acid sequence set forth in any one of SEQ ID NOS: 1-109, or
7) a peptide that comprises 15, 16, 17, 18, 19, 20, 21, 22, 23, 24, or 25 contiguous amino acids of the amino acid sequence set forth in any one of SEQ ID NOS:110-155.

2. The antiviral polypeptide of claim 1 which is capable of at least one antiviral activity that is selected from
(i) substantially impairing binding of a virus to a cell to which the virus exhibits tropism;
(ii) substantially impairing fusion of a virus to a cell membrane of a cell to which the virus exhibits tropism;
(iii) substantially impairing viral entry by a virus into a cell to which the virus exhibits tropism;
(iv) substantially impairing viral replication or viral assembly by a virus in a cell to which the virus exhibits tropism;
(v) substantially impairing release from a virus-infected cell of viral particles that have been synthesized in the cell as a result of infection by the virus; and
(vi) substantially impairing lysis of a virus-infected cell that results from infection of the cell by the virus.

3. A fusion protein which comprises the antiviral polypeptide of claim 1.

4. The antiviral polypeptide of claim 1 in which at least one amino acid situated at an identified amino acid sequence position in the amino acid sequence of the polypeptide comprises at least one of (i) a non-naturally occurring amino acid, or (ii) an amino acid that is not found at the identified amino acid sequence position in any naturally occurring homologue having at least 90% sequence identity to the antiviral polypeptide.

5. A pharmaceutical composition comprising the antiviral polypeptide of claim 1; and a pharmaceutical carrier or excipient.

6. A method of substantially impairing a viral activity in a cell, comprising contacting the cell with the antiviral polypeptide of claim 1, wherein the viral activity that is substantially impaired comprises at least one of:
(i) binding of a virus to a cell to which the virus exhibits tropism;
(ii) fusion of a virus to a cell membrane of a cell to which the virus exhibits tropism;
(iii) viral entry by a virus into a cell to which the virus exhibits tropism;
(iv) viral replication or viral assembly by a virus in a cell to which the virus exhibits tropism;
(v) release from a virus-infected cell of viral particles that have been synthesized in the cell as a result of infection by the virus; and
(vi) lysis of a virus-infected cell that results from infection of the cell by the virus.

7. The method of claim 6 in which the cell is contacted with the antiviral polypeptide in vitro.

8. A method of reducing likelihood or severity of viral infection in a subject, comprising administering to the subject a therapeutically effective amount of the pharmaceutical composition of claim 5.

9. A method for treating a subject having or suspected of being at risk for having a viral infection, comprising administering to the subject a therapeutically effective amount of the pharmaceutical composition of claim 5.
